(19) United States
(12) Patent Application Publication
(10) Pub. No.: US 2012/0330401 A1
(43) Pub. Date: Dec. 27, 2012

(54) STENT DELIVERY SYSTEM

(75) Inventors: Ryota Sugimoto, Ashigarakami-gun (JP); Takashi Kitaoka, Ashigarakami-gun (JP)

(73) Assignee: TERUMO KABUSHIKI KAISHA, Shibuya-ku (JP)

(21) Appl. No.: 13/608,594

(22) Filed: Sep. 10, 2012

Related U.S. Application Data
(63) Continuation of application No. PCT/JP2011/057209, filed on Mar. 24, 2011.

(30) Foreign Application Priority Data
Mar. 30, 2010 (JP) 2010-077679

(51) Int. Cl. A61F 2/84

(52) U.S. Cl. 623/1.12

(57) ABSTRACT

A stent delivery system includes a body and an operation unit disposed at the proximal end of the body. The body includes a self-expanding stent, an inner tube body, and a stent-accommodating tube body in which the stent is accommodated. The inner tube body has a stent-holding part enabling the stent to be re-accommodated into the stent-accommodating tube body. The operation unit includes a rack member fixed to a proximal end of the stent-accommodating tube body, an operation rotary roller having a working gear wheel that engages the teeth of the rack member, thereby causing the rack member to move forward and backward; and a connector fixed to a proximal end portion of a proximal-side tube that penetrates the stent-accommodating tube body and protrudes from the proximal end of the stent-accommodating tube body.

### CROSS REFERENCES TO RELATED APPLICATIONS

This application is a continuation of International Application No. PCT/JP2011/057209 filed on Mar. 24, 2011, and claims priority under 35 U.S.C. §119 to Japanese Patent Application No. 2010-077679 filed in the Japanese Patent Office on Mar. 30, 2010, the entire content of both of which is incorporated herein by reference.

### TECHNICAL FIELD

The present invention generally relates to a stent delivery system for use in improving a stenosis or an occluded part generated in a living-body lumen such as blood vessel, bile duct, trachea, esophagus, or urethra.

### BACKGROUND DISCUSSION

A stent delivery system, in general, has a stent for improving a stenosis or an occluded part. The stent is generally a tubular medical device which, for treating various diseases arising from stenosis or occlusion of a blood vessel or other living-body lumen, is used to dilate the stenosed or occluded part and indwelled there to secure an inner cavity.

The description will be made below taking a blood vessel as an example, which is a non-restrictive example.

A stent is a device which is small in diameter at the time of insertion into a living body from the outside, and which is expanded in a targeted stenosis or occluded part to increase in diameter and to maintain the lumen as it is.

In general, a stent has a cylindrical body formed from by processing metallic wires or a metallic pipe. The stent is mounted to a catheter or the like in a radially reduced state, is inserted into a living body, is expanded in a target part (stenosis or occluded part) by some method, and is fixed in secure contact with the inner wall of the lumen, thereby maintaining the lumen shape. Stents are classified, by function and indwelling method, into self-expandable stents and balloon-expandable stents. A balloon-expandable stent does not have an expanding function in itself. The stent mounted on a balloon is inserted into a target part, then the balloon is inflated, and the stent is expanded (plastically deformed) by dilation force of the balloon, whereby the stent is fixed in secure contact with the inner surface of the target lumen. This type of stent needs the just-mentioned stent-expanding operation. On the other hand, a self-expandable stent is provided with an expanding function of its own. The self-expanding stent is inserted into a living body in a radially reduced state, and is opened up in a target portion to spontaneously return into its original expanded state, thereby being fixed in secure contact with the inner wall of the lumen and maintaining the lumen shape.

The purpose of indwelling of a stent nowadays is mostly to return a blood vessel that is stenosed or occluded for some reason into its original patent state. In fact, most of the stents are used mainly for prevention or restraining of re-stenosis which might occur after such a procedure as PTCA. In recent years, to suppress the probability of re-stenosis more assuredly, drug-eluting stents carrying a drug such as immunosuppressant or carcinostatic agent have also been used, and their effects have been publicly known.

Many of the self-expandable stents are used in peripheral areas such as inferior limb or carotid artery, and include, for example, stents having a form as shown in International Application Publication No. WO96/26689 (JP-T-H11-505441).

In addition, International Application Publication No. WO2005/032614 (JP-T-2007-504897) discloses a system for delivery and deployment of a medical device (stent) into a patient's body, which has a delivery catheter including an inner catheter member having a region for attaching the medical device and an outer restraining member coaxially fitted over the inner catheter member and the medical device. In this delivery system, the outer restraining member is capable of movement in an axial direction relative to the inner catheter member, and a control handle which has a rotatable thumbwheel connected to a retraction mechanism is provided. The inner catheter member has a proximal end portion attached to the control handle, and the outer restraining member has a proximal end portion attached to the retraction mechanism. With the thumbwheel rotated, a rectilinear motion of the retraction mechanism is induced, an outer restraining member sheath is retracted toward the proximal end, and the medical device is exposed with the inner catheter member kept stationary.

In the stent delivery system using a self-expandable stent as in International Application Publication No. WO96/26689, the self-expanding property possessed by the stent makes it difficult to position the stent at the time of stent indwelling compared with the case of a balloon-expandable stent. Further, a jumping phenomenon may occur in which the stent jumps out from the stent delivery system. If this phenomenon occurs, the stent would be placed at a position deviated from the planned or intended placement position. In addition, there is a case where, after the stent is discharged to a certain extent during the stent indwelling procedure, readjustment of the indwelling position of the stent is needed. In the system as described in International Application Publication No. WO96/26689, however, re-accommodation of the stent into the stent delivery system is difficult to achieve.

In the stent delivery system disclosed in International Application Publication No. WO2005/032614, the operability of the outer restraining member for releasing the stent is good. Even in the stent delivery system in this international application publication, however, re-accommodation of the stent into the stent delivery system is difficult to perform.

A need thus exists for a stent delivery system using a self-expandable stent, in which a stent-releasing operation can be performed favorably, a stent can be re-accommodated into a stent-accommodating tube body even after the stent is exposed to a certain extent from the stent-accommodating tube body, and the operation of accommodating the stent into the stent-accommodating tube body is easy to carry out.

### SUMMARY

According to one aspect, a stent delivery system comprises: a stent delivery system main body and an operation unit, with the operation unit being disposed at a proximal end portion of the stent delivery system main body. The stent delivery system main body includes: a substantially cylindrically-shaped stent possessing a center axis and having a multiplicity of side-wall openings, with the stent being compressed toward its center axis upon insertion into a living body and being restorable to its pre-compression shape by expanding outward during indwelling in the living body, the stent possessing a proximal end portion; an inner tube body possessing a distal end portion, wherein the inner tube body includes a distal-side tube having a guide wire lumen, and a proximal-side tube connected to a proximal end portion of the distal-side tube; and a stent-accommodating tube body possessing a distal end portion, wherein the proximal-side tube penetrates the stent-accommodating tube body, and the stent is accommodated in the distal end portion of the stent-accommodating tube body. The stent covers the distal end portion of the inner tube body, and the stent is releasable by moving the stent-accommodating tube body in a proximal direction relative to the inner tube body. The operation unit includes a housing, a shaft-shaped rack member accommodated in the housing and fixed to a proximal end of the stent-accommodating tube body, with the rack member possessing teeth; a rotatably mounted operation rotary roller having a working gear wheel which engages the teeth of the rack member to move the rack member within the housing; and a connector fixed to a proximal end portion of the proximal-side tube and protruding proximally beyond the proximal end of the stent-accommodating tube body, the connector being held by the housing. The stent delivery system also includes means for releasably holding the proximal end portion of the stent to permit re-accommodation of the stent into the stent-accommodating tube body by forward movement of the stent-accommodating tube body after partial exposure of the stent from the stent-accommodating tube body. The stent is releasable from the stent-accommodating tube body by moving the rack member toward the connector through rotation of the operation rotary roller in one rotational direction and, after partial exposure of the stent from the stent-accommodating tube body, the stent is re-accommodated into the stent-accommodating tube body by moving the rack member within the housing away from the connector through rotation of the operation rotary roller in a direction reverse to the one rotational direction.

According to another aspect, a stent delivery system comprises: a distal-side tube possessing a guide wire lumen which opens at opposite ends to permit passage of a guide wire to guide the stent delivery system to a target site in a living body, wherein the distal-side tube possesses a proximal end portion; a proximal-side tube connected to the proximal end portion of the distal-side tube, with the proximal-side tube possessing a distal end portion; a stent-accommodating tube body surrounding at least a portion of the distal-side tube and the distal end portion of the proximal-side tube, with the stent-accommodating tube body possessing a distal end portion having an inner surface spaced outwardly from an outer surface of the portion of the distal-side tube so that a space exists between the outer surface of the portion of the distal-side tube and the inner surface of the distal end portion of the stent-accommodating tube body; and a stent accommodated in the space between the outer surface of the portion of the distal-side tube and the inner surface of the distal end portion of the stent-accommodating tube body so that the stent surrounds the portion of the distal-side tube and is covered by the distal end portion of the stent-accommodating tube body. The stent includes a side-wall provided with a plurality of through openings, and the stent is compressed inwardly while accommodated in the space and is covered by the distal end portion of the stent-accommodating tube body and being restorable to a pre-compression shape by expanding outwardly when the stent-accommodating tube is moved proximally relative to the distal-side tube to release the stent. An elongated rack member is positioned in a housing and is fixed to a proximal end of the stent-accommodating tube body, and a rotatably mounted operation roller operatively engages the rack member so that operative rotation of the operation roller moves the rack member relative to the housing to thus move the stent-accommodating tube body. A stent holder is positioned in the space between the outer surface of the portion of the distal-side tube and the inner surface of the distal end portion of the stent-accommodating tube body. The stent holder holds the proximal end portion of the stent so that when a distal end portion of the stent is exposed outside the stent-accommodating tube body and is no longer covered by the stent-accommodating tube body by virtue of the stent-accommodating tube body being moved in a proximal direction relative to the distal-side tube through rotation of the operation roller in one rotational direction, the exposed distal end portion of the stent is re-accommodated inside and covered by the distal end portion of the stent-accommodating tube body through rotation of the roller in a rotational direction opposite the one rotational direction.

By rotating the roller in the predetermined direction, the rack member is moved within the housing toward the connector, whereby the stent can be released from the stent-accommodating tube body. Therefore, a stent-releasing operation is rather easy to carry out. Further, after partial exposure of the stent from the stent-accommodating tube body, the stent can be re-accommodated into the stent-accommodating tube body by moving the rack member within the housing in the opposite direction through rotation of the roller in the direction reverse to the predetermined direction. Therefore, it is possible, even after the stent is exposed from the stent-accommodating tube body to a certain extent, to re-accommodate the stent into the stent-accommodating tube body. Thus, re-placement of the stent can be performed. In addition, the operation of accommodating the stent into the stent-accommodating tube body is relatively easy to conduct, since it is only necessary to rotate the roller.

A configuration is preferably adopted in which the proximal-side tube has a lumen a distal end portion of which opens in the stent-accommodating tube body and which provides communication to the proximal end of the proximal-side tube and in which liquid can be injected into the stent delivery system from the connector by using the lumen in the proximal-side tube, priming of the inside of the distal end portion of the stent-accommodating tube body is fairly easy to carry out. Further, liquid (for example, a drug) can be ejected from the distal end of the stent-accommodating tube body.

### BRIEF DESCRIPTION OF DRAWINGS

FIG. 1 is a partly omitted external appearance view of a stent delivery system according to an embodiment disclosed here by way of example.

FIG. 2 is an enlarged view of a distal end portion of the stent delivery system shown in FIG. 1.

FIG. 3 is an enlarged longitudinal cross-sectional view of the distal end portion of the stent delivery system shown in FIG. 1.

FIG. 4 is a partly omitted enlarged external appearance view of an inner tube body (including a stent) of the stent delivery system shown in FIG. 1.

FIG. 5 is an illustration for explaining the vicinity of the distal end portion of the stent delivery system shown in FIG. 1.

FIG. 6 is a partly omitted enlarged cross-sectional view of the distal end portion of the stent delivery system shown in FIG. 1.

FIG. 7 is an illustration for explaining an internal structure of the vicinity of an intermediate portion of the stent delivery system shown in FIG. 1.

FIG. 8 is a front view of an example of an in-vivo indwelling stent for use in the stent delivery system.

FIG. 9 is a development view of the in-vivo indwelling stent of FIG. 8.

FIG. 10 is an enlarged view of a proximal-end-side connection section of the in-vivo indwelling stent of FIG. 8.

FIG. 11 is a cross-sectional view taken along a section line XI-XI of FIG. 10.

FIG. 12 is an illustration for explaining an internal structure of an operation unit of the stent delivery system.

FIG. 13 is an enlarged front view of the operation unit of the stent delivery system.

FIG. 14 is a plan view of the operation unit of the stent delivery system shown in FIG. 13.

FIG. 15 is an illustration for explaining the internal structure of the operation unit of the stent delivery system.

FIG. 16 is an illustration for explaining the internal structure of the operation unit of the stent delivery system.

FIG. 17 is an illustration for explaining the internal structure of the operation unit of the stent delivery system.

FIG. 18 is an illustration for explaining an operation of the stent delivery system.

FIG. 19 is an illustration for explaining the operation of the stent delivery system.

FIG. 20 is an illustration for explaining the operation of the stent delivery system.

FIG. 21 is an illustration for explaining the operation of the stent delivery system.

FIG. 22 is an illustration for explaining the operation of the stent delivery system.

FIG. 23 is an enlarged longitudinal cross-sectional view of a distal end portion of a stent delivery system as another embodiment.

FIG. 24 is a development view of another example of the in-vivo indwelling stent for use in the stent delivery system.

FIG. 25 is an illustration for explaining a stent delivery system in which the in-vivo indwelling stent of FIG. 24 is used.

### DETAILED DESCRIPTION

An example of a stent delivery system (in other words, a body organ lesion improving instrument) disclosed here is described in detail below with reference to the accompanying drawing figures. The stent delivery system 1 includes a stent delivery system main body 2 and an operation unit 6 disposed at a proximal end portion of the stent delivery system main body 2. The stent delivery system main body 2 includes: a stent 10 having a multiplicity of side-wall openings, formed in a roughly cylindrical shape, compressed toward a center axis at the time of insertion into a living body, and capable of being restored into its pre-compression shape by expanding outward at the time of indwelling in the living body; an inner tube body 3 having a guide wire lumen 61; and a stent-accommodating tube body (stent-accommodating member) 5 which accommodates the stent 10 in a distal end portion thereof. In the stent delivery system main body 2, the stent 10 is so disposed as to cover a distal end portion of the inner tube body 3, and the stent 10 is releasable by moving the stent-accommodating tube body 5 in a proximal direction relative to the inner tube body 3. The operation unit 6 has a moving mechanism for moving the stent-accommodating tube body 5.

In addition, the inner tube body 3 includes a distal-side tube 31 having the guide wire lumen 61, and a proximal-side tube 34 connected to a proximal end side of the distal-side tube 31.

The operation unit 6 includes: a housing 40; a shaft-shaped rack member 43 accommodated in the housing 40 and fixed to a proximal end of the stent-accommodating tube body 5 (specifically, a proximal tube 22); an operation rotary roller 50 having a working gear wheel 54 which engages with teeth 66 of the rack member 43 and which is operable to move the rack member 43 within the housing 40; and a connector 46 which is fixed to a proximal end portion of the proximal-side tube 34 penetrating the stent-accommodating tube body 5 (specifically, the proximal tube 22) fixed to the rack member 43 and protruding beyond the proximal end of the stent-accommodating tube body 5 and which is held by the housing 40.

In addition, the stent delivery system 1 is configured 35, 36 to effect stent-holding to releasably hold the stent 10 and, after partial exposure of the stent 10 from the stent-accommodating tube body 5, enable re-accommodation of the stent 10 into the stent-accommodating tube body 5 by forward movement of the stent-accommodating tube body 5. Furthermore, with the operation rotary roller 50 rotated in a predetermined direction, the shaft-like rack member 43 is moved within the housing 40 toward the connector 46, whereby the stent 10 can be released from the stent-accommodating tube body 5. In addition, with the operation rotary roller 50 rotated in a direction reverse to the predetermined direction after partial exposure of the stent 10 from the stent-accommodating tube body 5, the shaft-like rack member 43 is moved within the housing 40 in a direction reverse to the direction toward the connector 46, whereby the stent 10 can be re-accommodated into the stent-accommodating tube body 5.

In the stent delivery system 1 in this embodiment, the proximal-side tube 34 of the inner tube body 3 has a lumen 38, and a distal end portion of the lumen 38 opens in the stent-accommodating tube body and provides communication to a proximal end of the stent-accommodating tube body. The proximal-side tube 34 is connected to a proximal end side of the distal-side tube 31 through a connection member. The stent delivery system 1 in this embodiment is configured to inject liquid into the stent delivery system from the connector 46 by using the lumen 38 in the proximal-side tube 34.

The stent delivery system 1 includes the operation unit 6 disposed at the proximal end portion of the stent delivery system main body 2. This example of the stent delivery system 1 shown in the drawings also includes: the stent 10 having the multiplicity of side-wall openings, formed in a roughly cylindrical shape, compressed toward the center axis (inward) at the time of insertion into a living body, and configured to be restored to its pre-compression shape by expanding outward at the time of indwelling in the living body. As noted, the inner tube body 3 is also provided with the guide wire lumen 61 and the stent-accommodating tube body 5 which accommodates the stent 10 in the distal end portion of the inner tube body. The stent 10 is so disposed as to cover the distal end portion of the inner tube body 3.

More specifically, the stent delivery system 1 includes: the stent 10 restorable into its pre-compression shape by expanding outward at the time of indwelling in the living body; the stent-accommodating tube body 5 which accommodates the stent 10 in the distal end portion of the stent delivery system 1, and the inner tube body 3 which slidably passes through the inside of the stent-accommodating tube body 5 and by which the stent 10 is released via the distal end of the stent-accommodating tube body 5. The stent 10 has a distal end portion oriented toward the distal end of the stent-accommodating tube body 5, and a proximal end portion oriented toward the proximal end of the stent-accommodating tube body 5. Further, the stent 10 does not substantially have any bent free end which is at least oriented toward the proximal end, other than the proximal end portion of the stent. After a distal-side portion of the stent 10 is exposed from the stent-accommodating tube body 5, the exposed portion can be re-accommodated into the stent-accommodating tube body 5 by moving the stent-accommodating tube body 5 in the distal direction. The guide wire lumen 61 of the stent delivery system 1 has one end opening at the distal end of the stent delivery system, and the other end opening on the proximal side relative to a stent-accommodating part of the stent-accommodating tube body 5.

The stent delivery system main body 2 includes: the stent 10; the stent-accommodating tube body 5 which accommodates the stent 10 in the distal end portion of the stent-accommodating tube body 5; and the inner tube body 3 slidably passing through the inside of the stent-accommodating tube body 5.

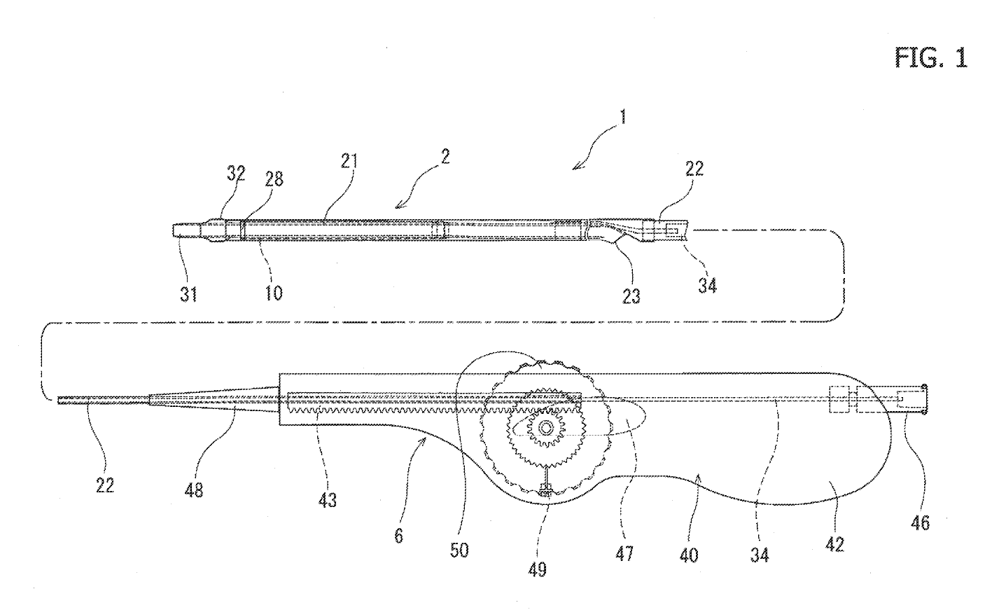

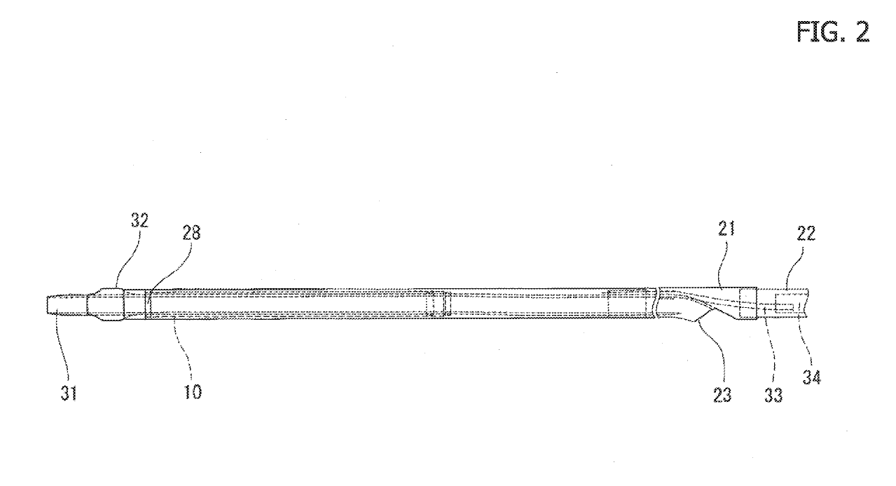

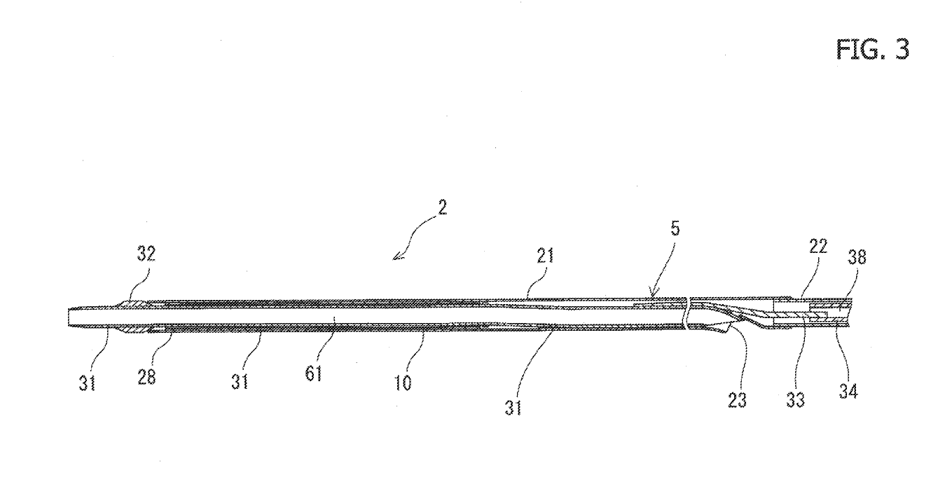

As shown in FIGS. 1 to 7, the stent-accommodating tube body 5 includes a distal tube 21, and the proximal tube 22 fixed to a proximal end of the distal tube 21.

The distal tube 21 is a tubular body, which is open at its distal end and at its proximal end. The distal opening is a release port for the stent 10 when the stent 10 is indwelled in a target portion of a lumen. The stent 10 is released via the distal opening, whereby it is relieved from a stress load, and expands to be restored to its pre-compression shape. A distal end portion of the distal tube 21 is the stent-accommodating part for accommodating the stent 10 in the inside of the distal end portion. In addition, the distal tube 21 has a side hole 23 disposed on the proximal side relative to the stent-accommodating part. The side hole 23 is a hole for leading out a guide wire to the exterior.

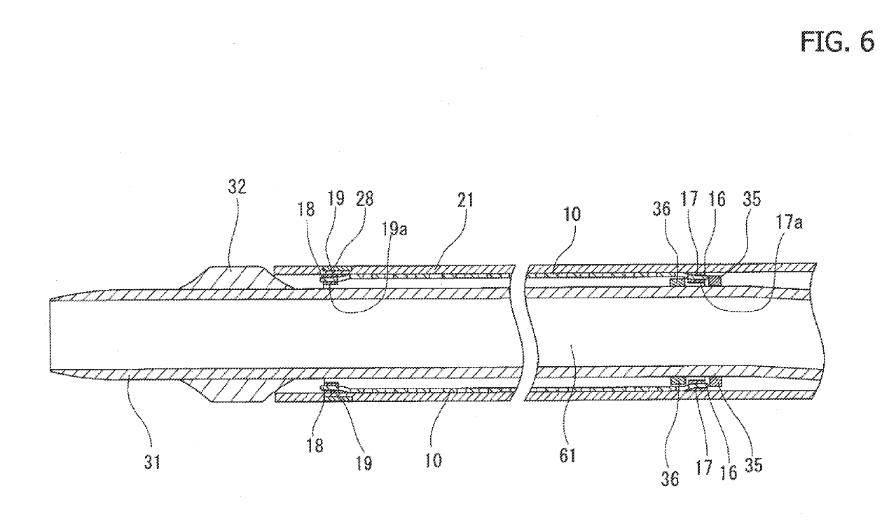

A radiopaque marker 28 is preferably disposed at the distal end portion of the distal tube 21. As shown in FIG. 6, the stent 10 is accommodated in the distal tube 21 in such a manner that the position of the distal end of the distal tube 21 substantially coincides with the position of the distal end of the radiopaque marker 28. The radiopaque marker 28 is preferably formed in a tubular shape, from a radiopaque material. As a material for forming the radiopaque marker, one element (simple substance) or two or more elements (alloy) selected from an element group consisting of iridium, platinum, gold, rhenium, tungsten, palladium, rhodium, tantalum, silver, ruthenium, and hafnium can be used suitably.

In addition, the proximal tube 22 is a tube body having a lumen penetrating it from the distal end of the proximal tube 22 to the proximal end of proximal tube 22. The distal end of the proximal tube 22 is fixed to the proximal end of the above-mentioned distal tube 21, and the proximal end portion of the proximal tube 22 is fixed to the rack member 43 accommodated in the operation unit 6 which will be described later.

The outside diameter of the distal tube 21 is preferably 0.5 to 4.0 mm, more preferably 0.8 to 2.0 mm, the inside diameter of the distal tube 21 is preferably 0.2 to 1.8 mm, and the length of the distal tube 21 is preferably 50 to 500 mm, more preferably 100 to 300 mm.

The outside diameter of the proximal tube 22 is preferably 0.3 to 4.0 mm, more preferably 0.5 to 1.0 mm, the inside diameter of the proximal tube 22 is preferably 0.1 to 1.0 mm, and the length of the proximal tube 22 is preferably 500 to 4,000 mm, more preferably 800 to 2,000 mm.

Materials for forming the distal tube 21 and the proximal tube 22 are selected taking into account physical properties (flexibility, hardness, strength, sliding property, anti-kinking property, stretchability) required of the tubes. Examples of preferable materials include stainless steel, superelastic metal, polyethylene, polypropylene, nylon, polyethylene terephthalate, fluoro polymer such as PTFE or ETFE, and thermoplastic elastomer. The thermoplastic elastomer is appropriately selected from nylon-based ones (e.g., polyamide elastomer), urethane-based ones (e.g., polyurethane elastomer), polyester-based ones (e.g., polyethylene terephthalate elastomer), and olefin-based ones (e.g., polyethylene elastomer, polypropylene elastomer).

The distal tube 21 is preferably more flexible than the proximal tube 22. Such a setting helps ensure good operability.

Furthermore, the outer surface of the stent-accommodating tube body 5 (the distal tube 21 and the proximal tube 22) is preferably subjected to a treatment for causing the outer surface to exhibit lubricity. Examples of such a treatment include a method in which the outer surface is coated with a hydrophilic polymer such as poly (2-hydroxyethyl methacrylate), polyhydroxyethyl acrylate, hydroxypropyl cellulose, methyl vinyl ether-maleic anhydride copolymer, polyethylene glycol, polyacrylamide, polyvinylpyrrolidone, and dimethylacrylamide-glycidyl methacrylate copolymer, or a method in which the hydrophilic polymer is fixed onto the outer surface. In addition, the inner surface of the distal tube 21 may be coated with the above-mentioned hydrophilic polymer or the hydrophilic polymer may be fixed onto the inner surface, for helping to ensure good slidability of the inner surface in relation to the stent 10 and the inner tube body 3.

As shown in FIGS. 1 to 7, the inner tube body 3 includes: the distal-side tube 31 of which a distal end portion protrudes beyond the distal end of the stent-accommodating tube body 5; the proximal-side tube 34; a wire-formed member 33 interconnecting a proximal end portion of the distal-side tube 31 and a distal end portion of the proximal-side tube 34; and the connector 46 fixed to the proximal end of the proximal-side tube 34.

In this embodiment, the inner tube body 3 has a proximal-side opening of the guide wire lumen which opens in a side portion on the proximal side relative to the stent-accommodating part of the stent-accommodating tube body 5. The stent-accommodating tube body 5 has the side hole disposed on the proximal side relative to the stent-accommodating part. A guide wire can be passed via the side hole and the proximal-side opening.

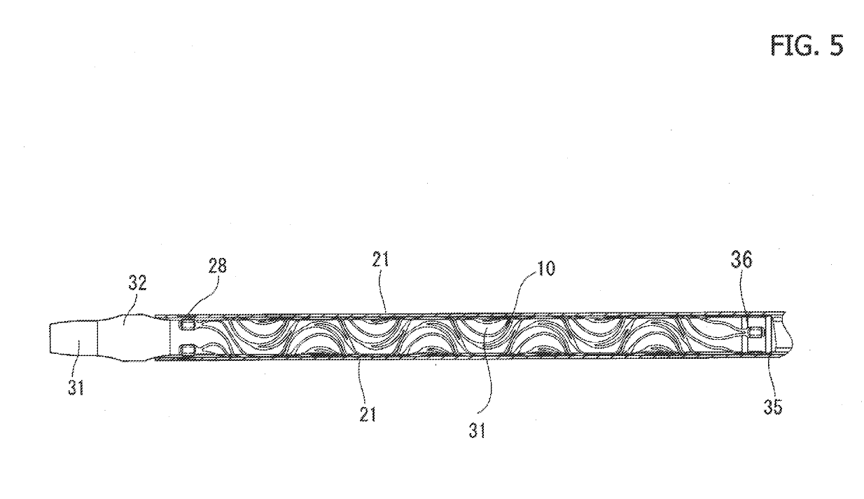

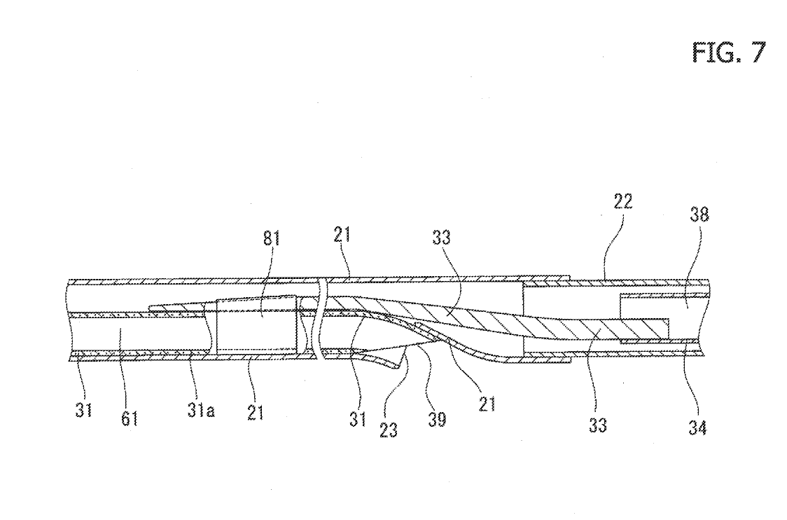

As shown in FIG. 5, the distal end of the distal-side tube 31 protrudes distally beyond the distal end of the stent-accommodating tube body 5 (the distal tube 21). In addition, the distal-side tube 31 is provided with a stopper 32 which inhibits movement of the stent-accommodating tube body 5 in the distal direction. As shown in FIG. 7, the proximal end portion of the distal-side tube 31 is curved, enters into the side hole 23 of the distal tube 21, and is disengageably engaged with the side hole 23 of the distal tube 21. The outside diameter of the distal-side tube 31 is preferably 0.2 to 2.0 mm. As shown in FIG. 5, a distal end portion of the distal-side stopper 32 is preferably decreased in diameter toward the distal end. The outside diameter at a greatest-diameter portion of the stopper 32 is preferably 0.5 to 4.0 mm. In addition, it is preferable that a proximal end portion of the stopper 32 is also decreased in diameter toward the proximal end, as shown in FIG. 5. The distal-side tube 31 has the guide wire lumen 61 extending from the distal end to the proximal end of the distal-side tube 31. The position of a proximal opening 39 of the guide wire lumen 61 is preferably located at a position deviated by 10 to 400 mm, particularly 50 to 350 mm, to the proximal side from the distal-most end of the distal-side tube 31. In addition, the position of the proximal opening 39 is preferably deviated by about 50 to 250 mm to the proximal side from the proximal-most end of the stent 10 (in other words, the proximal end of the stent-accommodating part).

The stent delivery system 1 includes the proximal-side tube 34 penetrating the stent-accommodating tube body 5, and with the stent-holding function by which the stent 10 is releasably held (preferably, the proximal end portion of the stent is releasably held) and by which it is ensures that, after partial exposure of the stent 10 from the stent-accommodating tube body 5, the stent 10 can be re-accommodated into the stent-accommodating tube body 5 by moving the stent-accommodating tube body 5 forward.

The stent delivery system 1 in this embodiment is configured so that the inner tube body 3 includes: a distal-side contact section 36 which is located inside the proximal end portion of the stent 10 at such a position as not to enter the side-wall openings of the stent 10; and a proximal-side contact section 35 which is provided at a position rearward of the proximal end of the stent 10 and in proximity to the distal-side contact section 36 and which can be brought into contact with the proximal end of the stent 10. The distal-side contact section 36 and the proximal-side contact section 35 are examples of means for releasably holding the stent 10 (proximal end portion of the stent), relative to the distal-side tube for example, so that after partial exposure of the stent 10 from the stent-accommodating tube body 5, such exposed portion of the stent can be re-accommodated inside the stent-accommodating tube body 5 by forward (distal) movement of the stent-accommodating tube body 5. The stent 10 is provided with a proximal-side inwardly projecting (proximal-end inwardly projecting) section 17a capable of making contact with the distal-side contact section 36 of the inner tube body 3. Furthermore, the stent 10 is so disposed that the proximal-side inwardly projecting section 17a is located between the distal-side contact section 36 and the proximal-side contact section 35 of the inner tube body 3. This configuration helps ensure that, after partial exposure of the stent 10 from the stent-accommodating tube body 5, the stent 10 can be re-accommodated into the stent-accommodating tube body 5 by forward movement of the stent-accommodating tube body 5.

The distal-side contact section 36 and the proximal-side contact section 35 constitute a stent holder possessing an annular shape and positioned in the space between the outer surface of the portion of the distal-side tube 31 and the inner surface of the distal end portion of the stent-accommodating tube body 5. The stent holder 35, 36 holds the proximal end portion of the stent 10 so that when the distal portion of the stent 10 is exposed outside the stent-accommodating tube body 5 by virtue of the stent-accommodating tube body 5 being moved in the proximal direction relative to the distal-side tube 31 as a result of rotary operation of the roller 50 in one rotational direction and resulting movement of the rack 43 such that the distal portion of the stent 10 is no longer covered by the stent-accommodating tube body 5, the exposed distal portion of the stent 10 can be re-accommodated inside and covered by the stent-accommodating tube body 5 through rotation of the roller 50 in a rotational direction opposite the one rotational direction and resulting movement of the rack. In the illustrated embodiment, at least a portion of the stent holder 35, 36 axially overlaps the proximal end portion of the stent 10.

The stent 10 used in this embodiment is a so-called self-expandable stent which has a multiplicity of openings in its side surface and which can be restored into its pre-compression shape by expanding outward at the time of indwelling in a living body. Further, the stent 10 used here has the distal end portion oriented toward the distal end of the stent-accommodating tube body 5 and the proximal end portion oriented toward the proximal end of the stent-accommodating tube body 5. Further, the stent 10 does not substantially have any bent free end at least oriented toward the proximal end, other than the proximal end portion. In addition, after the distal end portion of the stent 10 is exposed from the stent-accommodating tube body 5, the exposed distal end portion can be re-accommodated into the stent-accommodating tube body 5 by moving the stent-accommodating tube body 5 in the distal direction.

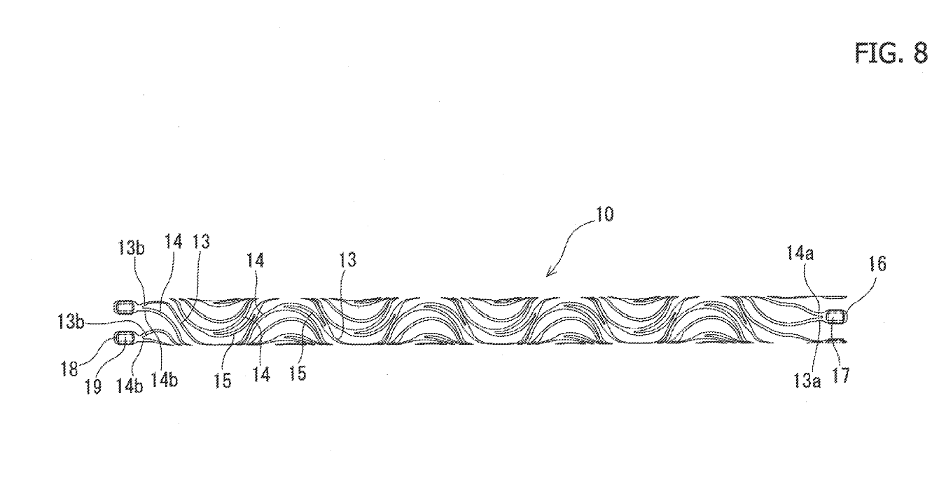

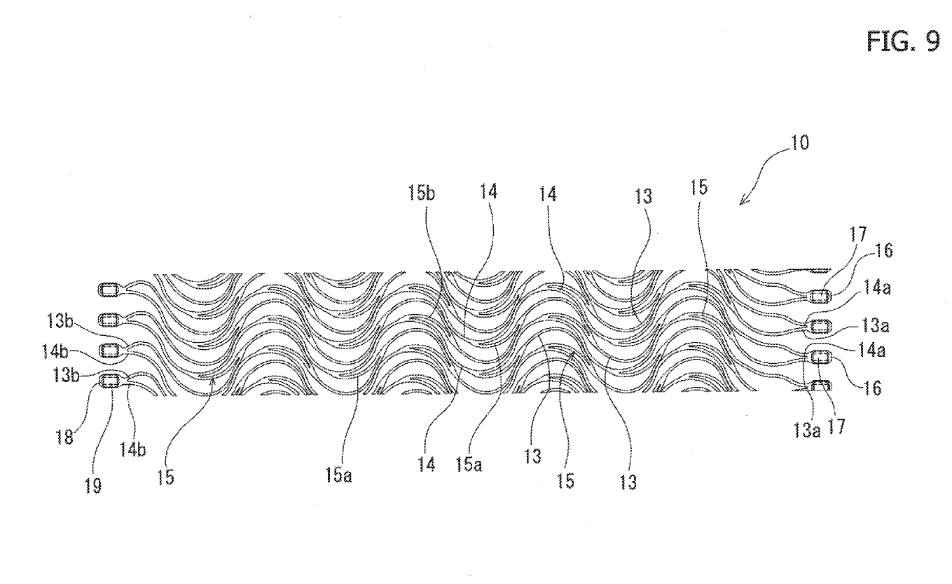

The stent to be used may be one in which an end portion of each filamentous component is connected to another filamentous component and which, therefore, does not have any free end. In addition, the stent to be used may be one as shown in FIGS. 8 and 9.

Generally speaking, the stent 10 includes wavy struts 13, 14 extending in the axial direction from one end to the other end of the stent and arranged in plural along a circumferential direction of the stent, and one or more link struts 15 interconnecting adjacent ones of the wavy struts and extending over a predetermined length along the axial direction. Furthermore, ends of the wavy struts 13, 14 are connected to ends of the adjacent wavy struts. In addition, the stent 10 has the multiple openings formed between the struts.

Particularly, the stent 10 shown in FIGS. 8 and 9 includes: first wavy struts 13 extending in the axial direction from one end to the other end of the stent 10 and arranged plural in number along the circumferential direction of the stent; second wavy struts 14 each located between the first wavy struts 13, extending in the axial direction from one end to the other end of the stent, and arranged plural in number along the circumferential direction of the stent; and one or more link struts 15 each interlinking an adjacent pair of a first wavy strut 13 and a second wavy strut 14, and extending over the predetermined length in the axial direction. In addition, vertexes of the second wavy strut 14 are shifted by a predetermined length along the axial direction of the stent from vertexes of the first wavy strut 13 proximate thereto in the circumferential direction of the stent 10 and curved to the same direction. End portions 13a, 13b of the first wavy strut 13 are coupled to end portions 14a, 14b of the second wavy strut proximate thereto.

The stent 10 in this embodiment is a so-called self-expandable stent which is formed in a roughly cylindrical shape, is compressed toward the center axis at the time of insertion into a living body, and is restored into its pre-compression shape by expanding outward at the time of indwelling in the living body.

The first wavy struts 13 extend in the axial direction substantially in parallel to the center axis of the stent. In addition, the first wavy struts 13 are arranged in plurality along the circumferential direction of the stent. The number of the first wavy struts 13 is preferably three or more, particularly three to eight. Further, the plurality of first wavy struts 13 are preferably arranged at roughly regular angular intervals around the center axis of the stent.

The second wavy struts 14 also extend in the axial direction substantially in parallel to the center axis of the stent. In addition, the second wavy struts 14 are arranged in plurality along the circumferential direction of the stent, and are each disposed between the first wavy struts. The number of the second wavy struts 14 is preferably three or more, particularly three to eight. Further, the plurality of second wavy struts 14 are preferably arranged at roughly regular angular intervals around the center axis of the stent. The number of the second wavy struts 14 is preferably the same as the number of the first wavy struts 13.

In addition, the stent 10 has the one or more link struts 15 each of which interconnects an adjacent pair of the first wavy strut 13 and the second wavy strut 14 and which extend over the predetermined length in the axial direction. Particularly, in the stent 10 in this embodiment, the link strut 15 has one end in the vicinity of an inflection point of the wavy strut on one side, has the other end in a region ranging from the vicinity of a vertex of the adjacent wavy strut on the other side to a position a little beyond the vertex, extends in the axial direction, and is curved to the same direction as the vertex of the wavy strut on the other side. As shown in FIG. 9, the link strut 15 is composed of first link struts 15a which are curved and have vertexes directed toward one side in the circumferential direction of the stent 10 and second link struts 15b which are curved and have vertexes directed toward the other side in the circumferential direction of the stent 10. In addition, the link strut 15 is curved in an arcuate shape, and has a radius approximately equal to that of an arc of a curved portion of the first wavy strut 13 or the second wavy strut 14 which is proximate thereto in the circumferential direction of the stent 10.

The stent 10 in this embodiment has coupling sections 16, 18 by which an end portion of every one of the first wavy struts 13 is coupled to an end portion of either of the proximate second wavy struts. Specifically, one-end-side end portion 13a of the first wavy strut 13 of the stent 10 is coupled to one-end-side end portion 14a of one of the second wavy struts 14 proximate to the first wavy strut 13 (specifically, the second wavy strut 14 which is proximate to, and located on the circumferential-directionally other side of, the first wavy strut 13) by the coupling section 16. In addition, the other-end-side end portion 13b of the first wavy strut 13 is coupled to other-end-side end portion 14b of one of the second wavy struts 14 proximate to the first wavy strut 13 (specifically, the second wavy strut 14 proximate to, and located on the circumferential-directionally one side of, the first wavy strut 13) by the coupling section 18. In other words, at the coupling section 16 on one end side and at the coupling section 18 on the other end side, the combinations of the first wavy strut 13 and the second wavy strut 14 coupled to each other are different (are shifted by one at a time).

In addition, as shown in FIG. 6, the stent 10 has the proximal-side inwardly projecting section 17a capable of making contact with the distal-side contact section 36 of the inner tube body 3. The stent 10 is so disposed that the proximal-side inwardly projecting section 17a is located between the distal-side contact section 36 and the proximal-side contact section 35 of the inner tube body 3. The proximal-side inwardly projecting section 17a is preferably composed of a radiopaque marker (radiopaque marker) 17 mounted to the proximal end portion (coupling section) 16 of the stent 10. As shown in FIG. 6, the proximal-side inwardly projecting section 17a of the stent 10 does not make contact with an outer surface of the distal-side tube 31 of the inner tube body 3. The proximal-side inwardly projecting section of the stent 10 may be composed of a thick wall section formed at the proximal end portion (coupling section) of the stent. The height of projection of the proximal-side inwardly projecting section is preferably 0.05 to 0.2 mm. In addition, the difference in height between the proximal-side inwardly projecting section of the stent and other non-projecting section is preferably 0.01 to 0.1 mm.

Furthermore, as shown in FIG. 6, the stent 10 in this embodiment may have a distal-side inwardly projecting section 19a at the distal end portion of the stent 10. The distal-side inwardly projecting section 19a is preferably composed of a radiopaque marker 19 mounted to the distal end portion (coupling section) 18 of the stent. The distal-side inwardly projecting section of the stent may be composed of a thick wall section formed at the distal end portion (coupling section) of the stent.

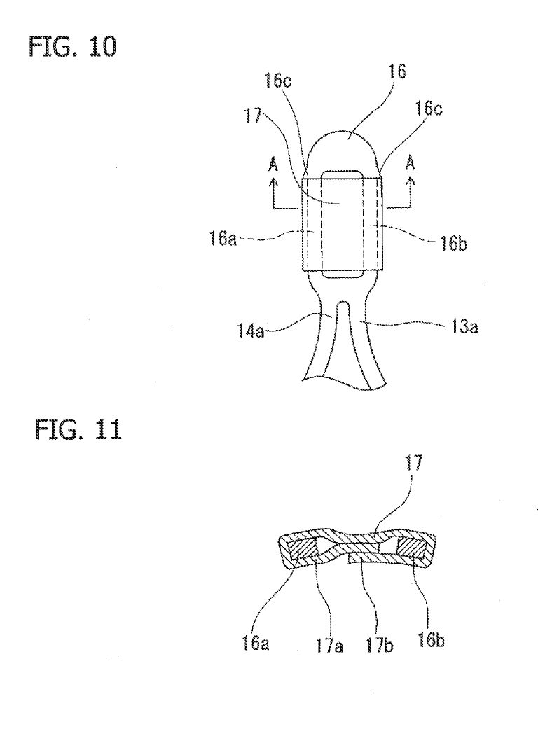

In the stent in this embodiment, the radiopaque marker 17 is attached to the coupling section 16. In this embodiment, the coupling section 16 has an opening, and has two frame sections 16a and 16b which extend in parallel in the direction toward the proximal end (end portion of a connecting section) of the stent, with a predetermined interval between the two frame sections 16a and 16b. The radiopaque marker 17 envelops substantially the whole part of the two frame sections 16a, 16b. In addition, the proximal-side inwardly projecting section 17a of the stent 10 is composed of a portion of the radiopaque marker 17 on the side of the inner surface of the stent. In the stent in this embodiment, as shown in FIGS. 10 and 11, the proximal-side inwardly projecting section 17a of the stent 10 is formed of a portion of a sheet-formed member wound around the opening of the proximal end portion (coupling section) 16 of the stent 10 on the side of the inner surface of the stent. Furthermore, in the stent in this embodiment, the sheet-formed member has an inner overlapping section 17b projecting to the side of the inner surface of the stent 10, to form a portion which projects more than other portions.

The radiopaque marker 17 forming the proximal-side inwardly projecting section preferably has a predetermined thickness (line diameter). In addition, in the configuration shown in FIGS. 10 and 11, the radiopaque marker 17 houses therein the two frame sections forming the proximal end portion (coupling section) 16, is hollowed in a central part thereof, and partly overlaps with itself, whereby it is fixed to the two frame sections.

The proximal end portion (coupling section) of the stent may not have any independent opening as shown in FIGS. 10 and 11. For instance, a configuration may be adopted in which the proximal end of the end portion 14a of the strut is continuous with an end portion of the frame section 16a while the proximal end of the end portion 13a of the strut is continuous with the frame section 16b, the opening is opened at an end portion thereof, and the opening communicates with a space between the two struts.

Further, the proximal end portion (coupling section) of the stent may be one that does not have the above-mentioned opening at all. In this type of stent, a coupling section is a plate-formed section having a predetermined area and being a little curved, and a radiopaque marker is so attached as to cover a face and a back face of the plate-formed section.

The proximal end portion of the stent is preferably provided with a lock section 16c for restraining movement in the proximal direction of the radiopaque marker 17 which forms the proximal-side inwardly projecting section of the stent 10. Particularly, it is preferable that two such lock sections 16c are disposed opposite to each other, as shown in FIG. 10. With such lock sections provided, it is ensured that, at the time of re-accommodation of the stent 10 into the stent-accommodating tube body 5, the radiopaque marker 17 can be prevented from being moved relative to or disengaged from the stent when the radiopaque marker 17 is pressed toward the proximal end of the stent by the distal-side contact section 36 of the inner tube body 3. In addition, the proximal end portion 16 of the stent is protruding in the proximal direction beyond the radiopaque marker 17. Therefore, at the time of releasing the stent, the proximal-side contact section 35 of the inner tube body 3 makes contact with the proximal end of the proximal end portion 16 of the stent 10, and the proximal-side contact section 35 of the inner tube body 3 does not make contact with the radiopaque marker 17. Accordingly, the radiopaque marker 17 would not be moved relative to or disengaged from the stent.

In all of the above-described embodiments, as the radiopaque marker, the above-mentioned sheet-formed member is preferably used, but one formed by winding a wire-formed member around the proximal end portion (coupling section) of the stent may also be used. Also in this case, further, it is preferable to provide an inner overlapping section which projects to the side of the inner surface of the stent. The material to be preferably used for forming the above-mentioned radiopaque marker is one element (simple substance) or two or more elements (alloy) selected from the element group consisting of iridium, platinum, gold, rhenium, tungsten, palladium, rhodium, tantalum, silver, ruthenium, and hafnium.

Fixation of the radiopaque marker can be carried out by any of welding, soldering, adhesion, fusing, and diffusion.

A material forming the stent 10 is preferably a superelastic metal. As the superelastic metal, a superelastic alloy is preferably used. The superelastic alloy here means a metal which is commonly called a shape-memory alloy and which exhibits superelasticity at least at a living body temperature (around 37° C.). Particularly preferable examples are such superelastic alloys as Ti—Ni alloy containing 49 to 53 at % of Ni, Cu—Zn alloy containing 38.5 to 41.5 wt % of Zn, Cu—Zn—X alloys (X=Be, Si, Sn, Al, or Ga) containing 1 to 10 wt % of X, and Ni—Al alloy containing 36 to 38 at % of Al. Especially preferred is the above-mentioned Ti—Ni alloy. In addition, mechanical properties can be appropriately modified by replacing part of the Ti—Ni alloy with 0.01 to 10.0 wt % of X to obtain Ti—Ni—X alloys (X=Co, Fe, Mn, Cr, V, Al, Nb, W, B or the like), or by replacing part of the Ti—Ni alloy with 0.01 to 30.0 at % of X to obtain Ti—Ni—X alloys (X=Cu, Pb, or Zr), or by selecting cold working ratio or/and final heat treatment conditions. The above-mentioned Ti—Ni—X alloys may be used and cold working ratio and/or final heat treatment conditions may be selected, whereby mechanical properties can be appropriately changed. The buckling strength (yield stress when loaded) of the superelastic alloy to be used is 5 to 200 kg/mm² (22° C.), more preferably 8 to 150 kg/mm², and the restoring stress (yield stress when unloaded) of the superelastic alloy is 3 to 180 kg/mm² (22° C.), more preferably 5 to 130 kg/mm². The superelasticity here means a property such that even if the material is subjected to deformation (bending, stretching, or compression) into a range for ordinary metals to be plastically deformed at use temperature, the material is restored substantially into its pre-compression shape without heating after release from the deformation.

In addition, the diameter of the stent when compressed is preferably 0.5 to 1.8 mm, more preferably 0.6 to 1.4 mm. The length of the stent when not compressed is preferably 5 to 200 mm, more preferably 8.0 to 100.0 mm. In addition, the diameter of the stent when not compressed is preferably 1.5 to 6.0 mm, more preferably 2.0 to 5.0 mm. Further, the material thickness of the stent is preferably 0.05 to 0.15 mm, more preferably 0.05 to 0.40 mm, and the width of the wavy struts is preferably 0.01 to 1.00 mm, more preferably 0.05 to 0.2 mm. Surfaces of the wavy struts have been preferably smoothened, more preferably been smoothened by electropolishing. In addition, the strength in radial direction of the stent is preferably 0.1 to 30.0 N/cm, more preferably 0.5 to 5.0 N/cm.

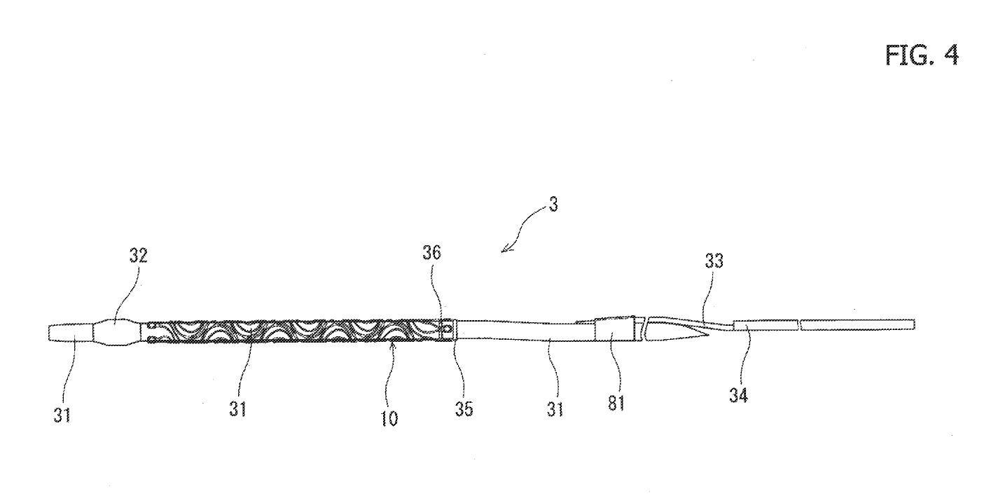

As shown in FIGS. 4, 5, and 6 (particularly, in FIG. 6), the inner tube body 3 has: the distal-side contact section 36 which is located in the proximal end portion of the stent 10 and which does not enter the side-wall openings of the stent 10; and the proximal-side contact section 35 which is disposed at a position rearward of the proximal end of the stent 10 and proximate to the distal-side contact section 36 and which is able to make contact with the proximal end of the stent 10. In addition, in this embodiment, as shown in FIGS. 4, 5, and 6 (especially, in FIG. 6), the distal-side contact section 36 is a distal-side projecting section projecting from the outer surface of the distal-side tube 31; like the distal-side contact section 36, the proximal-side contact section 35 is also a proximal-side projecting section projecting from the outer surface of the distal-side tube 31.

The proximal-side inwardly projecting section 17a of the stent 10 mentioned above is capable of making contact with the distal-side contact section 36 of the inner tube body 3. In addition, as shown in FIG. 6, the proximal-side inwardly projecting section 17a of the stent 10 is located between the distal-side contact section 36 and the proximal-side contact section 35 of the inner tube body 3.

As shown in FIGS. 5 and 6, the stent delivery system 1 in this embodiment has the distal-side contact section 36 at a position deviated by a predetermined distance toward the proximal end from the distal end of the distal-side tube 31. The distal-side contact section 36 is disposed at a position which is inside the proximal end portion of the stent 10 and which is deviated a little toward the distal end relative to the proximal end of the stent 10. In addition, the proximal-side contact section 35 is disposed at a position which is deviated a little toward the proximal end relative to the distal-side contact section 36. The proximal-side contact section 35 is disposed in the vicinity of and rearward (proximal side) of the proximal end of the stent 10. The proximal-side inwardly projecting section 17a of the stent 10 is located between the distal-side contact section 36 and the proximal-side contact section 35 of the inner tube body 3. Therefore, the distance between the distal-side contact section 36 and the proximal-side contact section 35 is slightly longer than an axial length of the proximal-side inwardly projecting section 17a of the stent 10. The distance between the distal-side contact section 36 and the proximal-side contact section 35 is preferably longer than the axial length of the proximal-side inwardly projecting section 17a of the stent 10 by 0.02 to 1.0 mm, more preferably by 0.05 to 0.3 mm.

In addition, the distal-side contact section 36 does not enter the side-wall openings of the stent 10. The distal-side contact section 36 is preferably an annular projecting section disposed continuously over the outer circumference of the distal-side tube 31. The annular projecting section is formed, for example, by attaching a tubular member to the outer circumference of the distal-side tube. With such an annular projecting section, assured contact of the annular projecting section with the proximal-side inwardly projecting section 17a of the stent 10 is realized. In addition, the distal-side contact section 36 is preferably a section which substantially does not make contact with the inner surface of the stent 10. This helps prevent the distal-side contact section 36 from constituting an obstacle at the time of releasing the stent. The distal-side contact section 36 has such a height that it can make contact with the proximal-side inwardly projecting section 17a of the stent 10. The height of the distal-side contact section 36 is preferably 0.06 to 0.11 mm, more preferably 0.08 to 0.11 mm. An axial length of the distal-side contact section 36 is preferably 0.1 to 3.0 mm, more preferably 0.3 to 2.0 mm.

While the distal-side contact section 36 is preferably an annular projecting section disposed continuously over the outer circumference of the distal-side tube 31, it may be composed of a plurality of discontinuous ribs arranged in an annular pattern.

In addition, the proximal-side contact section 35 is preferably an annular projecting section disposed continuously over the outer circumference of the distal-side tube 31. The annular projecting section is formed, for example, by attaching a tubular member to the outer circumference of the distal-side tube. The proximal-side contact section 35 is preferably a section which does not make contact with the inner surface of the stent-accommodating tube body 5. This helps prevent the proximal-side contact section 35 from obstructing an operation at the time of releasing the stent. The proximal-side contact section 35 has such a height that it can make contact with the proximal end of the stent 10. The height of the proximal-side contact section 35 is preferably 0.08 to 0.18 mm, more preferably 0.1 to 0.16 mm. In addition, an axial length of the proximal-side contact section 35 is preferably 0.1 to 3.0 mm, more preferably 0.3 to 2.0 mm. The distance between the outer surface of the proximal-side contact section 35 and an inner surface of the stent-accommodating tube body 5 is preferably 0.01 to 0.04 mm. In addition, it is preferable that the proximal-side contact section 35 is greater in height than the distal-side contact section 36, and that the difference in height between these sections is 0.02 to 0.1 mm.

Further, the distal-side contact section 36 and the proximal-side contact section 35 are preferably formed from a radiopaque material. As the radiopaque material, the materials for forming the radiopaque marker as above-mentioned can be used suitably. Particularly, it is preferable for these contact sections to be each formed by attaching a tubular member formed of the radiopaque material. Furthermore, it is preferable that the distal-side contact section 36 and the proximal-side contact section 35 are formed from the radiopaque material, and that they are different from each other in axial length. This helps facilitate rather easy discrimination between them. While which one of them is longer does not matter, the difference between them in axial length is preferably 0.3 to 1.0 mm.

In the stent delivery system 1 in this embodiment, the inner tube body 3 (specifically, the distal-side tube 31) has the opening 39 which communicates with the guide wire lumen on the proximal side relative to the stent-accommodating part of the stent-accommodating tube body 5.

In addition, the distal-side tube 31 may have a reinforcement layer 31a at least along a portion located on the proximal side relative to the proximal end of the stent, as shown in FIG. 7. The reinforcement layer 31a is preferably disposed over the whole part of the distal-side tube 31. A structure may be adopted in which the reinforcement layer 31a is not disposed at a distalmost portion of the distal-side tube 31. The reinforcement layer 31a is preferably a network-formed reinforcement layer. The network-formed reinforcement layer is preferably formed from braids. The braids can be formed, for example, from metallic wire of stainless steel, elastic metal, superelastic alloy, shape-memory alloy or the like having a wire diameter of 0.01 to 0.2 mm, preferably 0.03 to 0.1 mm. The braids may be formed from synthetic fiber such as polyamide fiber, polyester fiber, and polypropylene fiber.

As mentioned above and as shown in FIGS. 1 to 7, the inner tube body 3 includes the distal-side tube 31, the proximal-side tube 34, the wire-formed member 33 interconnecting the proximal end portion of the distal-side tube 31 and the distal end portion of the proximal-side tube 34, and the connector 46 fixed to the proximal end of the proximal-side tube 34.

The proximal-side tube 34 of the inner tube body is a tube having the penetrating internal lumen which has its distal end opening in the stent-accommodating tube body 5 (specifically, in a distal end portion of the proximal tube 22 of the stent-accommodating tube body 5) and has its proximal end opening in the connector 46.

In addition, as shown in FIG. 7, the inner tube body 3 has the connection member which interconnects the distal-side tube 31 and the proximal-side tube 34. In this embodiment, the connection member is composed of a wire-formed member 33 and a heat-shrinkable tube 81. A proximal end portion of the wire-formed member 33 enters the distal end portion of the proximal-side tube 34, and is fixed there. A distal end portion of the wire-formed member 33 is fixed to a side surface of the distal-side tube 31 by the heat-shrinkable tube 81. Further, in this embodiment, the wire-formed member 33 has a smaller diameter section on the distal side of a portion fixed to the distal-side tube 31.

The length of the inner tube body 3 is preferably 400 to 2,500 mm, more preferably 400 to 2,200 mm. In addition, the outside diameter of the proximal-side tube 34 is preferably 0.3 to 3.0 mm, more preferably 0.5 to 1.0 mm. The inside diameter of the proximal-side tube 34 is preferably 0.1 to 2.5 mm, more preferably 0.2 to 2.0 mm. The length of the distal-side tube 31 is preferably 10 to 400 mm, more preferably 50 to 350 mm. The outside diameter of the distal-side tube 31 is preferably 0.2 to 2.0 mm, more preferably 0.4 to 1.7 mm. In addition, the inside diameter of the lumen 61 is preferably 0.1 to 1.8 mm, more preferably 0.3 to 1.0 mm.

A material for forming the inner tube body (the distal-side tube 31 and the proximal-side tube 34) is preferably a material which has hardness and a certain degree of flexibility. Examples of the material which can be used suitably include stainless steel, superelastic metal, polyethylene, polypropylene, nylon, polyethylene terephthalate, fluoro-polymers such as ETFE, PEEK (polyether ether ketone), and polyimide. An outer surface of the inner tube body 3 may be coated with a biocompatible material, particularly an antithrombogenic material. Examples of the antithrombogenic material which can be used suitably include polyhydroxyethyl methacrylate, and hydroxyethyl methacrylate-styrene copolymer (for example, HEMA-St-HEMA block copolymer).

Furthermore, a part of the inner tube body 3 which may protrude beyond the stent-accommodating tube body 5 preferably has a lubricating outer surface. For this purpose, the outer surface of the inner tube body 3 may be coated with a hydrophilic polymer such as poly (2-hydroxyethyl methacrylate), polyhydroxyethyl acrylate, hydroxypropyl cellulose, methyl vinyl ether-maleic anhydride copolymer, polyethylene glycol, polyacrylamide, polyvinylpyrrolidone, and dimethylacrylamide-glycidyl methacrylate copolymer, or the hydrophilic polymer may be fixed to the outer surface. In addition, the whole outer surface of the inner tube body 3 may be coated with the just-mentioned hydrophilic polymer, or the hydrophilic polymer may be fixed to the outer surface. Further, an inner surface of the inner tube body 3 may also be coated with the just-mentioned hydrophilic polymer, or the hydrophilic polymer may be fixed to the inner surface, for enhancing the sliding properties of the inner surface for a guide wire.

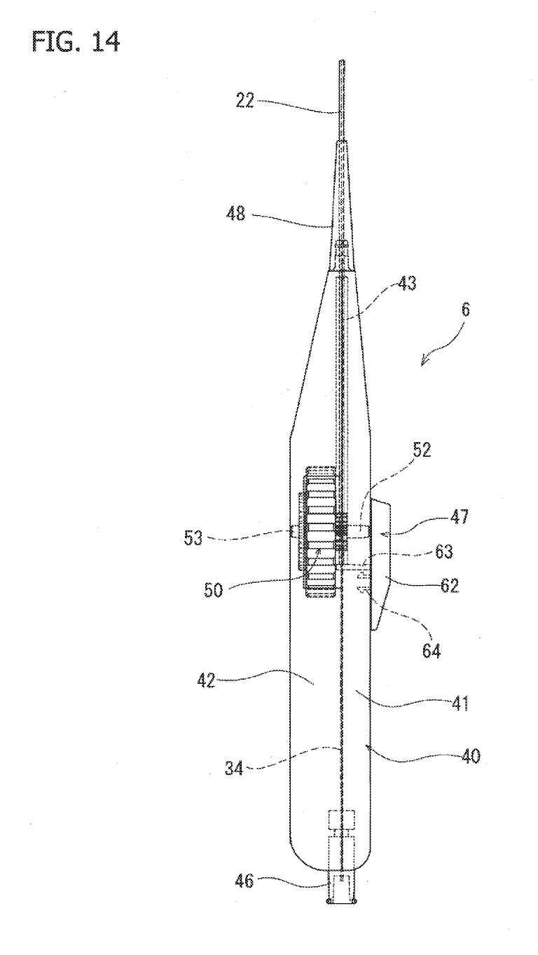

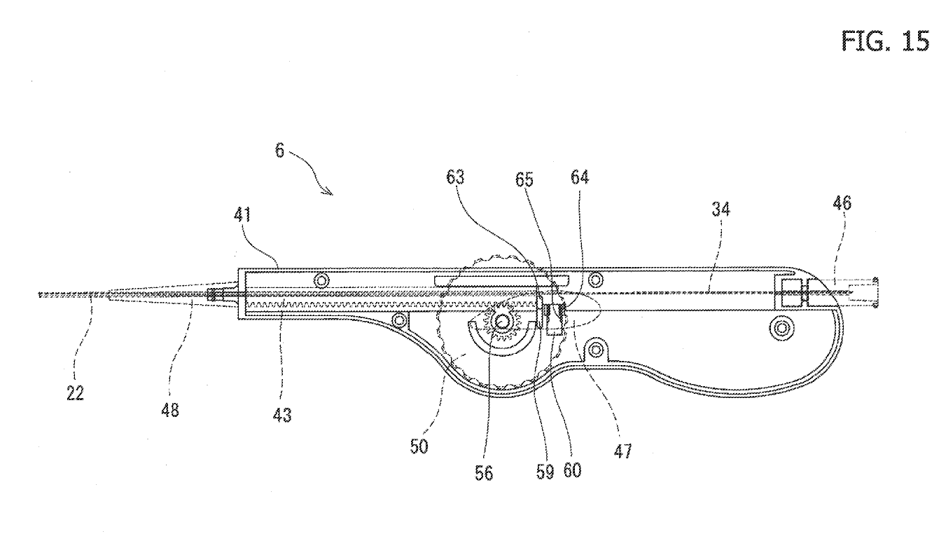

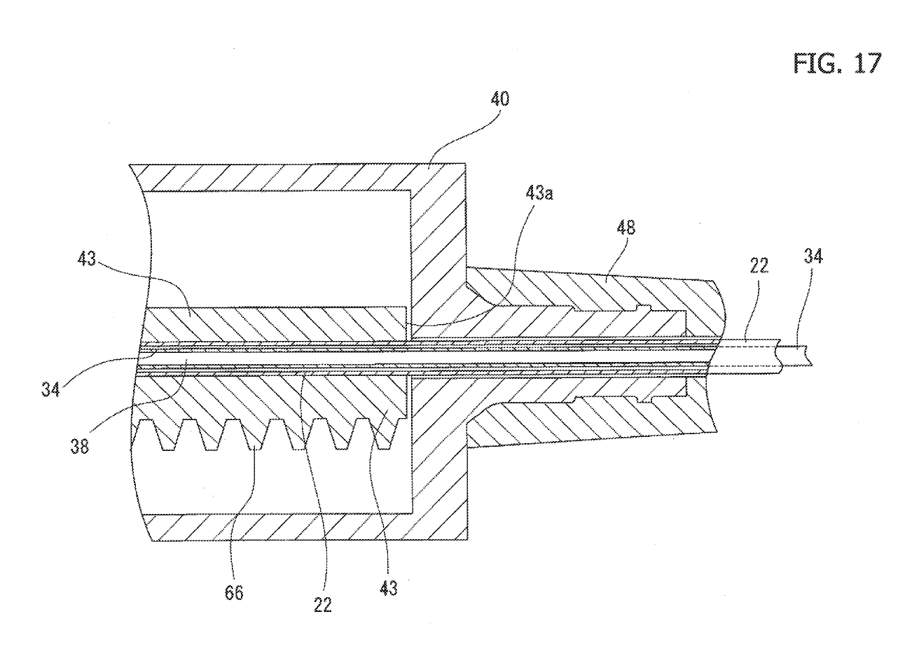

In addition, the proximal-side tube 34 penetrates the stent-accommodating tube body and protrudes beyond the proximal opening of the stent-accommodating tube body 5 (the proximal tube 22). As shown in FIGS. 1 and 13 to 19, the connector 46 is firmly attached or fixed to the proximal end portion of the proximal-side tube 34.

In the stent delivery system 1, a liquid-injecting device can be connected to the connector 46. Liquid injected by the liquid-injecting device thus connected passes through the lumen 38 inside the proximal-side tube 34, and flows out in a distal-side portion of the stent delivery system (the stent-accommodating tube body), whereby the inside of the stent delivery system (the stent-accommodating tube body) is flushed. Furthermore, the liquid can be ejected via the distal end of the stent delivery system (the stent-accommodating tube body).

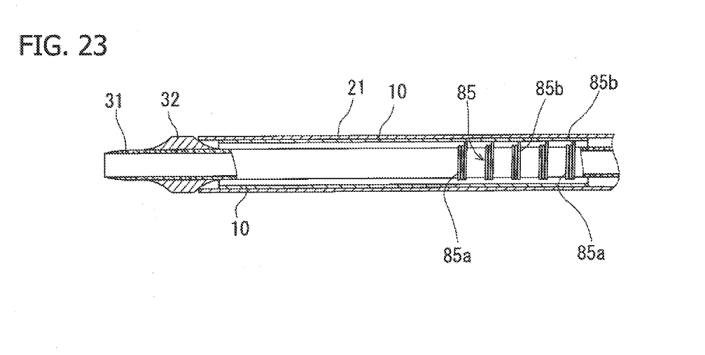

In addition, the stent-holding function for enabling the stent 10 possessed by the stent delivery system 1 to be re-accommodated into the stent-accommodating tube body 5 by forward movement of the stent-accommodating tube body 5 after partial exposure of the stent 10 from the stent-accommodating tube body 5 is not restricted to the one configured as above-described. For instance, the stent-holding ability can be exhibited by the configuration shown in FIG. 23 as another example.

In the configuration according to this embodiment, the inner tube body 3 has an elastic member 85 which is disposed at least on the outer surface of the inner tube body 3 located inside (radially inside) the proximal end portion of the stent and which presses the stent 10 outwardly toward the stent-accommodating tube body 5. The stent 10 is gripped between the elastic member 85 and the stent-accommodating tube body 5, and is slidable relative to the stent-accommodating tube body 5. In addition, the stent 10 is substantially non-slidable relative to the elastic member 85.

The elastic member 85 constitutes a stent holder and is fixed onto the outer surface of the inner tube body 3 (specifically, the distal-side tube 31). As shown in FIG. 23, the elastic member 85 is a wire coil which has a fixation section 85a for fixation to the distal-side tube 31 and an elastic section 85b for pressing the stent 10. The elastic member is annular-shaped. The fixation section 85a is formed by winding around the distal-side tube 31a wire constituting the wire coil. As shown in FIG. 23, the elastic section 85b is configured by a method in which the wire forming the fixation section 85a is spaced apart from the distal-side tube 31 while being enlarged in diameter. That is, both the fixation section 85a and the elastic section 85b can be formed by winding a wire around the distal tube 31, with the part of the wire constituting the fixation section 85a being wound more tightly and the part of the wire constituting the elastic section 85b being wound more loosely. The elastic section 85b has such a size and such a spring elasticity as to be able to press (apply an outwardly directed force to) the stent 10 accommodated in the stent-accommodating tube body 5. In addition, in this embodiment, at least the elastic section 85b of the elastic member 85 composed of the wire coil is preferably a little inclined toward the proximal end, as shown in FIG. 23. At least the elastic section 85b of the elastic member 85 composed of the wire coil may be a little inclined toward the distal end. Where the elastic section 85b of the elastic member 85 composed of the wire coil is thus inclined, better pressing of the stent 10 is ensured. In this embodiment, the elastic member 85 presses a part of the inner circumference of the stent 10 as shown in FIG. 23.

The stent delivery system can include a plurality of such elastic members 85 forming a stent holder which axially overlaps a proximal end portion of the stent. All of the elastic members 85 are disposed within the proximal end portion of the stent 10. The elastic members 85 are arranged at substantially regular (i.e., the same) intervals. But it is also possible for elastic members to be arranged so that the interval between axially adjacent elastic members 85 decreases along the direction toward the proximal end of the stent. The elastic member(s) 85 represents another example of means for releasably holding the stent 10 (proximal end portion of the stent), relative to the distal-side tube 31 for example, so that after partial exposure of the stent 10 from the stent-accommodating tube body 5, such exposed portion of the stent can be re-accommodated inside the stent-accommodating tube body 5 by forward (distal) movement of the stent-accommodating tube body 5.

Aspects of the operation unit 6 of the stent delivery system 1 disclosed by way of example will now be described. The operation unit 6 generally includes the moving mechanism for moving the stent-accommodating tube body 5. In this embodiment, the operation unit 6 includes: the housing 40; the shaft-like rack member 43 which is accommodated in the housing 40 and which is fixed to the proximal end of the stent-accommodating tube body 5 (specifically, the proximal tube 22); the operation rotary roller 50 having the working gear wheel 54 which engages the teeth 66 of the rack member 43 and which moves the rack member 43 within the housing 40; and the connector 46 fixed to the proximal end portion of the proximal-side tube 34 which penetrates the stent-accommodating tube body 5 (specifically, the proximal tube 22) fixed to the rack member 43 and which protrudes beyond the proximal end of the stent-accommodating tube body 5.

The operation unit 6 in this embodiment is configured to include a moving mechanism allowing the shaft-like rack member 43 to move backward and forward, whereby the stent-accommodating tube body 5 fixed to the rack member 43 can be withdrawn or moved rearwardly in the proximal direction to expose the stent and can be advanced or moved forwardly in the distal direction to once again accommodate the stent relative to the inner tube body 3.

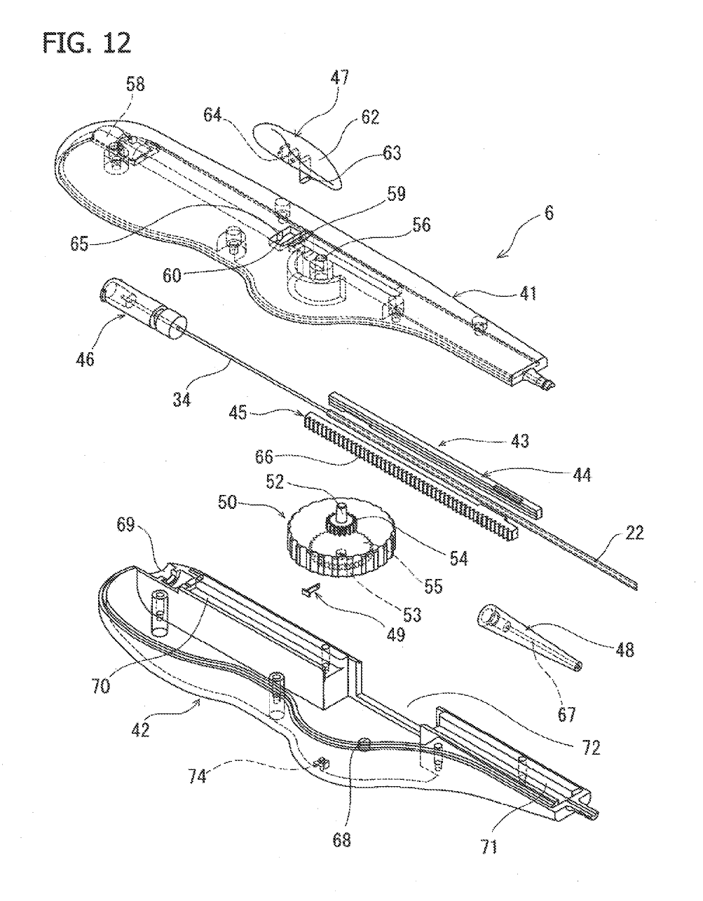

As shown in FIGS. 12 to 19, the housing 40 of the operation unit 6 is composed of a first housing portion 41 and a second housing portion 42. The housing 40 has a shape which is bent and rounded at a proximal end side and at a central portion, which enables relatively easy gripping, and which enables easy operation of the roller in a condition where the housing 40 is gripped.

As shown in FIGS. 12 to 19, the housing 40 also has: an opening 72 for permitting the operation rotary roller 50 to partially protrude from an accommodating section; a bearing section 56 for accommodating one end 52 of a rotary shaft of the roller 50; and a bearing section 68 for accommodating the other end 53 of the rotary shaft of the roller 50.

In addition, as shown in FIGS. 12 to 19, the connector 46 is tubular-shaped and is fixed to the proximal end portion of the proximal-side tube 34 of the inner tube body 3, and the housing 40 has an accommodating section 58, 69 for the connector 46. The shaft-shaped rack member 43 is fixed to a proximal end portion of the proximal tube 22 of the stent-accommodating tube body 5, and the housing 40 has a rack member accommodating section 70, 71 in which the rack member 43 is accommodated in an axially movable manner. In addition, a distal member 48 to be fitted over the proximal tube 22 of the stent-accommodating tube body 5 so as to permit sliding of the proximal tube 22 is fixed to a distal end portion of the housing 40. The distal member 48 has an internal passage 67 which the proximal tube 22 penetrates in a slidable manner.

As shown in FIG. 12, the rack member 43 is composed of a first rack member 44 and a second rack member 45, which are fixed to the proximal end portion of the proximal tube 22 of the stent-accommodating tube body 5 by clamping the proximal end portion of the proximal tube 22 between the first and second rack members 44. In addition, the rack member 43 is shaft-shaped and is longer than the stent 10 by a predetermined length. The rack member 43 has the teeth 66 formed on the side surface facing the roller 50 (a surface facing the lower side of the housing). The teeth 66 are formed on the whole part of a tooth-formed surface of the rack member 43.

As shown in FIGS. 12 to 19, the operation rotary roller 50 includes one end 52 of the rotary shaft disposed at a side surface on one side, the other end 53 of the rotary shaft disposed at a side surface on the other side, and the working gear wheel 54 which engages the teeth 66 of the rack member 43 to move the rack member 43 within the housing 40. The working gear wheel 54 is smaller in outer diameter than the outer diameter of the rotary roller. The outside diameter of the working gear wheel 54 is preferably 10 to 60 mm. In addition, the roller 50 has a gear-formed section 55 disposed for enabling intermittent rotation of the roller. The operation rotary roller 50 is partially exposed via the opening 72 of the housing 40, and the exposed part constitutes an operation part.

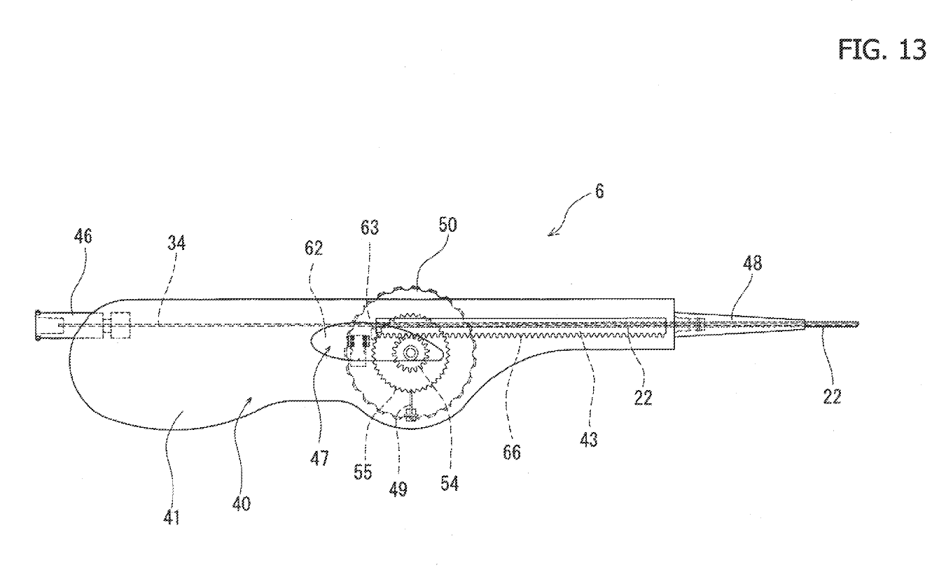

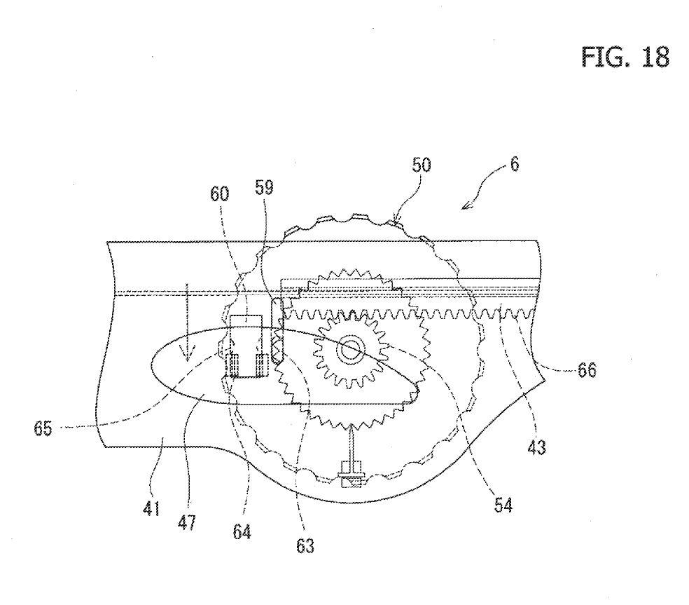

The operation unit 6 in this embodiment has a lock mechanism for releasably locking the rack member 43. The operation unit 6 has a lock lever 47. As shown in FIG. 12, the lock lever 47 includes a lock lever main body 62, a lock bar 63 protruding from the lock lever main body 62, and a mounting section 64 for mounting the lock lever 47 onto the housing. In addition, as shown in FIG. 12, the first housing 41 has a lock bar accommodating port 59 in which the lock bar 63 is slidably accommodated, and a mounting port 60 in which is slidably accommodated the mounting section 64. The mounting part 60 engages a distal end portion of the mounting section 64 and holds the lock lever 47. The mounting port 60 has a rib 65 for holding the lock lever 47 in a locked-state position and in an unlocked-state position. As shown in FIGS. 13 to 15, in a condition where the lock lever 47 is located on the side of the opening 72 of the housing 40, the lock bar 63 makes contact with a proximal end face of the rack member 43, thereby inhibiting the rack member 43 from moving backward (a direction toward the connector member 46; a stent-releasing direction). As shown in FIG. 18, when the lock lever 47 is pressed down (pressed in a direction for moving away from the opening 72 of the housing 40), the lock lever mounting section 64 rides over the rib 65 formed on an inner surface of the mounting port 60, and slides downward (in a direction moving away from the rack member 43) within the mounting port. The lock bar 63 also slides within the lock bar accommodating port 59 to move in the direction for coming away from the rack member 43. Consequently, the lock bar 63 comes out of contact with the proximal end face of the rack member 43, whereby locking is removed, and the roller 50 is permitted to be rotated. The unlocked state is held by the rib 65 formed on the inner surface of the mounting port 60.

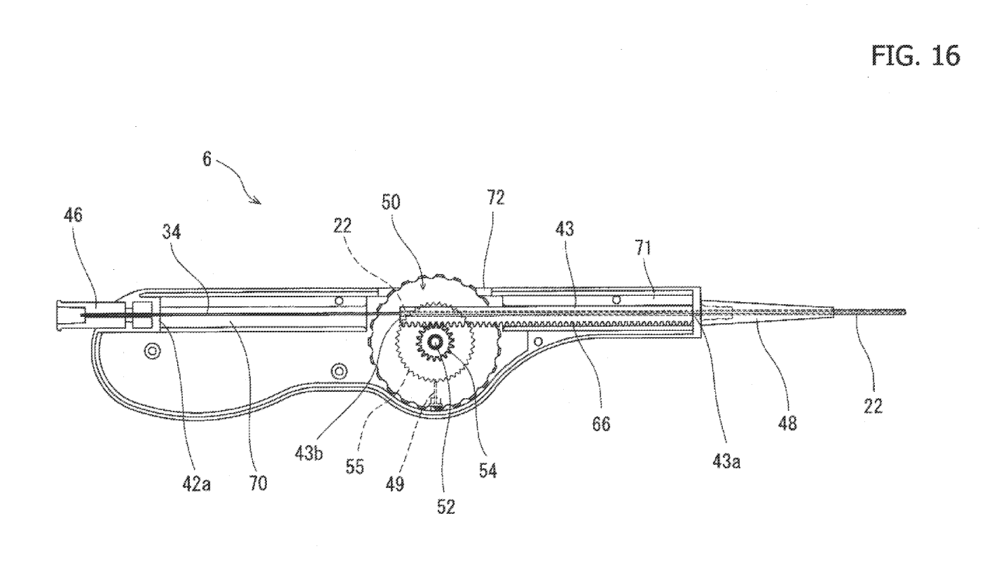

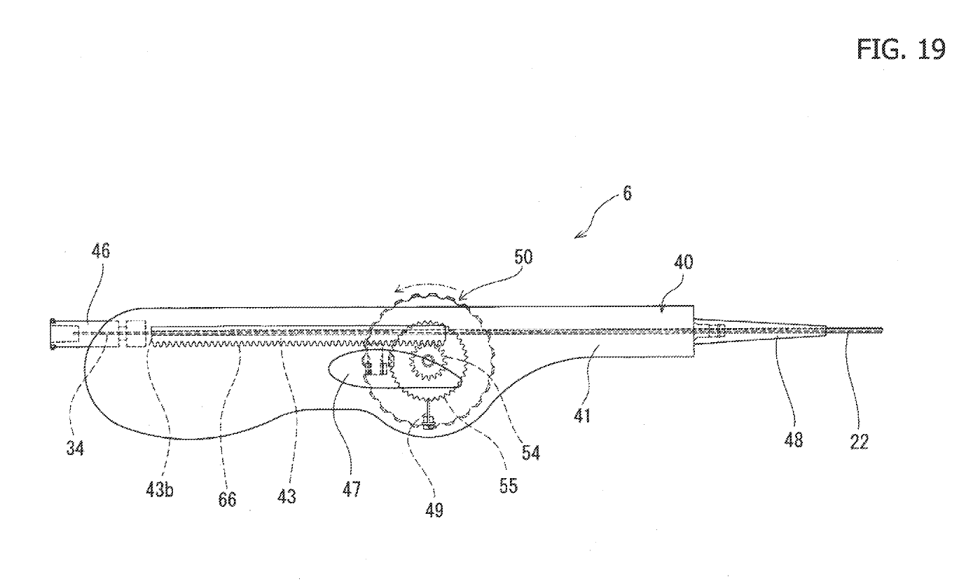

Further, the operation unit 6 in this embodiment has a movement-restraining section (rotation-restraining section) which, at the time of rotation of the operation rotary roller 50, makes contact with an end portion of the rack member to restrain movement of the rack member (in other words, rotation of the roller, or movement of the stent-accommodating tube body 5 relative to the inner tube body 3) in excess of a predetermined extent. Specifically, as shown in FIG. 16 which illustrates a condition where the first housing 41 has been detached from the operation unit 40, a distal end 43a of the rack member 43 makes contact with an inner surface of a distal end portion of the housing 40 (the second housing), and the rack member 43 is unable to advance further. Thus, the operation unit 6 in this embodiment has the movement-restraining section (rotation-restraining section) which, when the operation rotary roller 50 is rotated in the reverse direction to the predetermined direction (in an advancing direction of the stent-accommodating tube body 5; in a stent-accommodating direction), makes contact with the distal end 43a of the rack member 43 to thereby restrain further movement of the rack member (further rotation of the roller). Similarly, as shown in FIGS. 16 and 19, a proximal end 43b of the rack member 43 is able to make contact with an inner surface 42a of a proximal end portion of the housing 40 (the second housing), and the rack member 43 is unable to retreat further. Thus, the operation unit 6 in this embodiment has the movement-restraining section (rotation-restraining section) which, when the operation rotary roller 50 is rotated in the predetermined direction (in a retreating direction of the stent-accommodating tube body 5; in a stent-releasing direction), makes contact with the proximal end 43b of the rack member 43 to thereby restrain the rack member from moving (the roller from rotating) further (in excess of a predetermined extent).

In order to prevent the stent-accommodating tube body from being deformed or broken by an excessive force at the time of movement of the stent-accommodating tube body 5, a configuration may be adopted in which when a force in excess of a safety setpoint is exerted, engaging between the working gear wheel of the rotary roller and the teeth of the rack member is released, resulting in idling. Such an idling-generating mechanism can be formed, for example, by clearances in the bearing section 56 in which the one end 52 of the rotary shaft of the operation rotary roller 50 is accommodated and in the bearing section 68 in which the other end 53 of the rotary shaft of the roller 50 is accommodated. In addition, the above-mentioned safety setpoint is preferably set lower than the breaking strength of the stent-accommodating tube body.

Further, the operation unit 6 in this embodiment has a roller intermittent rotation mechanism which holds the operation rotary roller 50 to impart a rotation resistance and which permits intermittent rotation of the roller. In this embodiment, the roller intermittent rotation mechanism is composed of the gear-formed section 55 disposed on the operation rotary roller 50, and an elastically deformable pin 49 having a distal end portion entering a recess of the gear-formed section 55. The elastically deformable pin 49 has a body part extending in a direction toward the gear-formed section 55 and a proximal part disposed at a proximal end portion of the body part. The proximal part is fixed to a pin-fixing section 74 of the second housing 42. In this embodiment, the gear-formed section 55 is formed on a surface, different from a surface formed with the working gear wheel 54, of the roller 50. In addition, the roller 50 is pressed in a direction toward the opening 72 by the deformable pin 49, thereby being restrained from unprepared rotation. At the time of rotation of the roller 50, the pin 49 is deformed to permit rotation of the roller, and, when the distal end of the pin 49 enters the recess of the gear-formed section 55, a roller-holding state is attained, so that intermittent (stepwise) rotation of the roller is enabled. Furthermore, the pin 49 is preferably one that generates a sound upon restoration from a deformed state, during rotation of the roller. This enables an operator to recognize the rotation. Further, the pin 49 preferably generates rotational sounds discernible depending on the rotating direction of the roller. This makes it possible to confirm, by the sound, the direction in which the rotation is being carried out, in other words, whether the stent-accommodating tube body 5 is being moved forward or backward.

The operation of the stent delivery system disclosed by way of example here will be described below with reference to FIGS. 18 to 22.

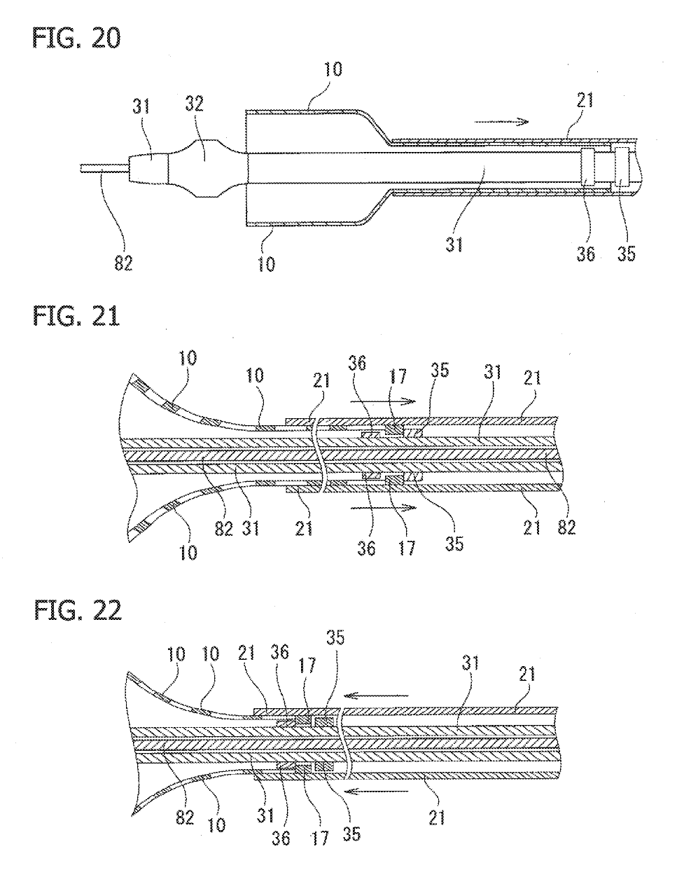

The stent delivery system 1 having a guide wire 82 passed through the distal-side tube 31 is inserted into a blood vessel to be treated, and the stent is brought to a target part. In this state, the whole part of the stent 10 is accommodated in the stent-accommodating tube body 5. Next, as shown in FIG. 18, the lock lever 47 is depressed, to unlock the rack member 43. Then, as shown in FIG. 19, the roller is rotated in the predetermined direction (in a direction of the illustrated arrow), whereby the stent-accommodating tube body 5 is moved backward relative to the inner tube body 3. This results in the stent 10 being gradually exposed and permitted to expand, starting from the distal end side thereof, as shown in FIG. 20. In the stent delivery system in this embodiment, movement of the stent 10 in the proximal direction is restrained by the contact of the proximal end of the stent 10 with the proximal-side contact section 35 of the inner tube body 3 (the distal-side tube 31) as shown in FIG. 21; therefore, the stent 10 can be exposed. The stent 10 exposed from the stent-accommodating tube body 5 tends to expand by the self-expanding force so as to be restored to its pre-compression shape. Thereafter, in a case where readjustment of the placing position of the stent 10 is needed, the roller is rotated in the reverse direction to the predetermined direction (the direction of the arrow). As a result, as shown in FIG. 22, the stent-accommodating tube body 5 is moved in the distal direction, and part of or the whole part of the stent is re-accommodated into the stent-accommodating tube body 5 (the distal-side tube 31). In this embodiment, movement of the stent 10 in the distal direction is restrained by the contact of the proximal-side inwardly projecting section 17a of the stent 10 with the distal-side contact section 36 of the inner tube body 3 (the distal-side tube 31); therefore, the stent 10 can be accommodated.

Then, after the stent is re-placed into an appropriate position, the roller is again rotated in the predetermined direction (the direction of the arrow), whereby the stent-accommodating tube body 5 is moved toward the proximal end, and the stent 10 is exposed from the distal opening of the stent-accommodating tube body 5. The roller is rotated in the predetermined direction (the direction of the arrow) until the proximal end of the stent is exposed, whereby the stent is released completely from the stent-accommodating tube body, and is disengaged from the inner tube body 3. In addition, in the stent delivery system 1 in this embodiment, the liquid-injecting device (not shown) can be connected to the connector 46, and a liquid can be injected into the stent delivery system 1 by the liquid-injecting device thus connected. The liquid injected via the connector 46 passes through the lumen 38 inside the proximal-side tube 34, and flows out in the distal-side portion of the stent delivery system (the stent-accommodating tube body), whereby the inside of the stent delivery system (the stent-accommodating tube body) can be flushed. Further, the liquid can also be ejected via the distal end of the stent delivery system (the stent-accommodating tube body).

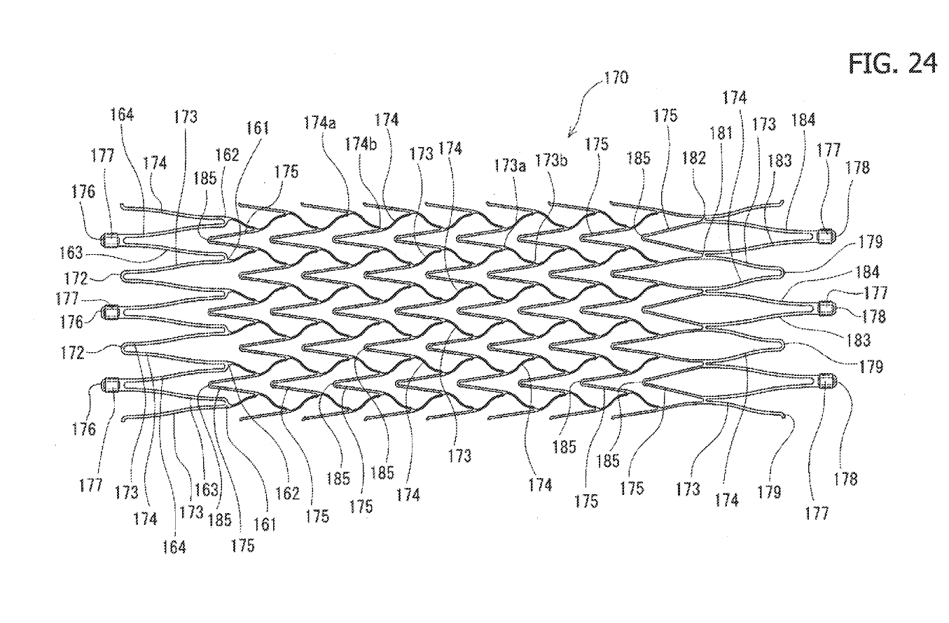

The stent used in the stent delivery system disclosed here may also be one as shown in FIG. 24. Like the above-described stent 10, this stent 170 is a so-called self-expandable stent which has a multiplicity of openings in its side surface and is capable of being restored into its pre-compression shape by expanding outward at the time of indwelling in a living body. Further, the stent 170 has a distal end portion oriented toward the distal end of the stent-accommodating tube body (stent-accommodating member) 5 and a proximal end portion oriented toward the proximal end of the stent-accommodating tube body (stent-accommodating member) 5. Furthermore, the stent 170 substantially does not have any bent free end which is at least oriented toward the proximal end, other than the proximal end portion thereof. That is, the stent 170 substantially does not have any bent ends which are unconnected (i.e., free end) and which are oriented toward the proximal end, other than the proximal end portion of the stent. After the distal end portion of the stent 170 is exposed from the stent-accommodating tube body 5, the exposed distal end portion can be re-accommodated into the stent-accommodating tube body 5 by moving the stent-accommodating tube body 5 toward the distal end relative to the inner tube body 3.

This stent 170 is an in-vivo indwelling stent formed in a roughly cylindrical shape. The stent 170 includes wavy struts 173, 174 extending in the axial direction from one end to the other end of the stent 170 and arranged in plurality along the circumferential direction of the stent, and a plurality of link struts 175 each interconnecting adjacent ones of the wavy struts 173, 174. The adjacent wavy struts 173, 174 have pluralities of close portions and open portions. Each link strut 175 interconnects a close portion of the adjacent wavy struts 173, 174, and is provided at its central portion with a bent section 185 oriented toward the distal end in the axial direction of the stent.

Particularly, in this stent 170, the plurality of wavy struts 173, 174 include a plurality of first wavy struts 173 each having a plurality of upper points 173a and a plurality of lower points 173b, and a plurality of second wavy struts 174 each having a plurality of upper points 174a and a plurality of lower points 174b and each located between the first wavy struts. Each adjacent pair of the first wavy strut 173 and the second wavy strut 174 are so arranged that the upper points or lower points of one of the two wavy struts and the lower points or upper points of the other of the two wavy struts adjacent thereto are set to substantially face each other, thereby forming the close portions. Each link strut 175 interconnects an upper point 173a or a lower point 173b of the first wavy strut 173 and a lower point 174b or an upper point 174a of the second wavy strut which constitute the close portion. In addition, each adjacent pair of the first wavy strut 173 and the second wavy strut 174 are so arranged that the lower points or upper points of one of the two wavy struts and the upper points or lower points of the other of the two wavy struts adjacent thereto are set to substantially face each other, thereby forming the open portions.

This stent 170 is a so-called self-expandable stent which is formed in a roughly cylindrical shape, is compressed toward a center axis thereof at the time of insertion into a living body, and is restored into its pre-compression shape by expanding outward at the time of indwelling in the living body.

The first wavy struts 173 extend in the axial direction substantially parallel to the center axis of the stent. In addition, the first wavy struts 173 are arranged plural in number along the circumferential direction of the stent. The number of the first wavy struts 173 is preferably two or more, more preferably three to eight. Further, the plurality of first wavy struts 173 are preferably arranged at substantially regular angular intervals around the center axis of the stent.

In this stent 170, the first wavy strut 173 has a series of substantially the same waveform over a predetermined length, exclusive of both end portions. Specifically, the first wavy strut 173 has a series of waves having substantially the same waveform, namely, same wavelength and same amplitude, over its portion other than the vicinity of both end portions. In a case where the first wavy strut 173 has the same waveform over substantially the whole part thereof, the wavelength, which varies depending on an outside diameter of the stent, is preferably 0.5 to 8.0 mm, particularly preferably 2.0 to 4.0 mm, and the amplitude is preferably 0.1 to 10.0 mm, particularly preferably 0.3 to 3.0 mm.

The second wavy struts 174 also extend in the axial direction substantially parallel to the center axis of the stent. The second wavy struts 174 are arranged plural in number along the circumferential direction of the stent, with each of the second wavy struts 174 being arranged between the first wavy struts. The number of the second wavy struts 174 is preferably two or more, more preferably three to eight. Further, the plurality of second wavy struts 174 are preferably arranged at substantially regular angular intervals around the center axis of the stent. In addition, the number of the second wavy struts 174 is equal to the number of the first wavy struts.

In this stent 170, the second wavy strut 174 has a series of substantially the same waveform over a predetermined length, exclusive of both end portions. Specifically, the second wavy strut 174 has a series of waves having substantially the same waveform, namely, the same wavelength and same amplitude, over its portion other than the vicinity of both end portions. In a case where the second wavy strut 174 has the same waveform over substantially its entirety, the wavelength, which varies depending on the outside diameter of the stent, is preferably 0.5 to 8.0 mm, more preferably 2.0 to 4.0 mm, and the amplitude is preferably 0.1 to 10.0 mm, more preferably 0.3 to 3.0 mm.

Furthermore, in this stent 170, the first wavy strut 173 and the second wavy strut 174 are substantially the same in waveform. Specifically, in this stent 170, the first wavy strut 173 and the second wavy strut 174 have substantially the same wavelength and substantially the same amplitude. In addition, the second wavy struts 174 are positionally shifted from the first wavy struts 173 by about a half of the wavelength along the axial direction of the stent.

consequently, as shown in FIG. 24, the first wavy strut 173 and the second wavy strut 174 adjacent to each other are so situated that the upper points 173a or lower points 173b of the first wavy strut 173 are substantially opposed to the lower points 174b or upper points 174a of the second wavy strut 174, whereby the close portions and the open portions are formed. In other words, in this stent 170, the first wavy strut 173 and the second wavy strut 174 adjacent to each other are so situated that their respective upper portions are not opposed to each other and their respective lower portions are not opposed to each other; therefore, the close portions and the open portions are provided alternately along the axial direction.

In addition, in the stent in this embodiment, the wavy struts 173, 174 are all the same in length, exclusive of both end portions. Therefore, when the stent is compressed inwardly toward its center axis, the struts approach one another in parallel to the axial direction. Since all the wavy struts are the same in length, the stent is favorably compressed radially without stiffing in the axial direction. In addition, in the stent in this embodiment, the wavy struts 173 and 174 are arranged at regular angular intervals around the center axis of the stent, exclusively of both ends thereof. This helps ensure that, when the stent is compressed toward its center axis, gaps between the struts are reduced in an even manner, so that favorable compression can be achieved without any overlapping of the struts.

The stent 170, as shown in FIG. 24, includes the link struts 175 which each interconnect the close portions of the adjacent wavy struts 173, 174, and has at its central portion the bent section 185 oriented toward the distal end in the axial direction of the stent. The axial length of the link strut 175, which varies depending on the outside diameter of the stent, is preferably 0.1 to 3.0 mm, more preferably 0.5 to 2.0 mm. In addition, each of the link struts 175 is symmetrical about the center axis of the stent 170 and about the vertex of the bent section 185. In this stent 170, substantially all the plurality of close portions of the first wavy strut 173 and the second wavy strut 174 adjacent to each other are interconnected by the link struts 175. In addition, the bent sections 185 of the link struts 175 are located in the vicinity of the open portion formed between the wavy struts 173, 174. The bent section 185 of the link strut 175 is a free end oriented toward the distal end of the stent 170. In addition, in the stent 170 in this embodiment, the link struts 175 are disposed in plural numbers and in series along the axial direction of the stent. The link struts 175 are disposed in plural numbers along the circumferential direction of the stent.

In addition, the stent 170 in this embodiment has, at the distal end portion of the stent: bent sections 172 each formed by coupling a distal end portion of the first wavy strut 173 and a distal end portion of the second wavy strut 174; bent sections 176 each formed by coupling a distal end portion of a filamentous part 163 connected to the first wavy strut 173 via a branching section 161 and a distal end portion of a filamentous part 164 connected to the second wavy strut 174 via a branching section 162. The bent sections 172 and the bent sections 176 are disposed alternately along the circumferential direction. A radiopaque marker 177 is attached to each of the bent sections 176. In addition, the bent sections 176 each having the radiopaque marker 177 are located on the more distal side of the stent than the bent sections 172.

The stent 170 has, at the proximal end portion of the stent: bent sections 179 each formed by coupling a proximal end portion of the first wavy strut 173 and a proximal end portion of the second wavy strut 174; bent sections 178 each formed by coupling a proximal end portion of a filamentous part 183 connected to the first wavy strut 173 via a branching section 181 and a proximal end portion of a filamentous part 184 connected to the second wavy strut 174 via a branching section 182. The bent sections 179 and the bent sections 178 are disposed alternately along the circumferential direction. In other words, in the stent 170, the bent sections 178 and the bent sections 179 form the proximal end portion oriented toward the proximal end of the stent-accommodating tube body. In addition, the radiopaque marker 177 is attached to each of the bent sections 178. In this stent 170, the radiopaque marker 177 forms a proximal-side inwardly projecting section 177a which will be described later. The bent sections 178 each having the radiopaque marker 177 are located on the more proximal side of the stent than the bent sections 179. In addition, the stent 170 does not have any free end oriented to the proximal end of the stent, other than the bent sections 178, 179. Therefore, when the stent-accommodating tube body is moved toward the distal end relative to the inner tube body after the distal end portion of the stent is partially exposed from the stent-accommodating tube body, the stent would not be caught on the stent-accommodating tube body since the stent does not have any free end oriented toward the stent-accommodating tube body. Consequently, re-accommodation of the stent into the stent-accommodating tube body (stent-accommodating member) can be achieved. In this stent 170, the filamentous parts 183 and 184 constituting the bent sections 178 of the proximal-side portion are longer along the axial direction than the filamentous parts 163 and 164 constituting the bent sections 176 of the distal-side portion. The stent 170 is inserted into a living body, starting from the distal end side (the side of the bent sections 176), to be indwelled.

In addition, the radiopaque marker 177 envelops part of or substantially the whole part of two frame sections constituting the bent section. The radiopaque marker 177 has a thin rectangular parallelepiped shape, houses the two frame sections therein, and is hollowed at a central portion thereof, whereby it is fixed to the two frame sections. As a material for forming the radiopaque marker, one element (simple substance) or two or more elements (alloy) selected from the element group consisting of iridium, platinum, gold, rhenium, tungsten, palladium, rhodium, tantalum, silver, ruthenium, and hafnium can be used suitably. In addition, the length of the marker is preferably 0.1 to 4.0 mm, more preferably 0.3 to 1.0 mm. The thickness of the marker is preferably 0.01 to 0.30 mm, more preferably 0.03 to 0.10 mm.

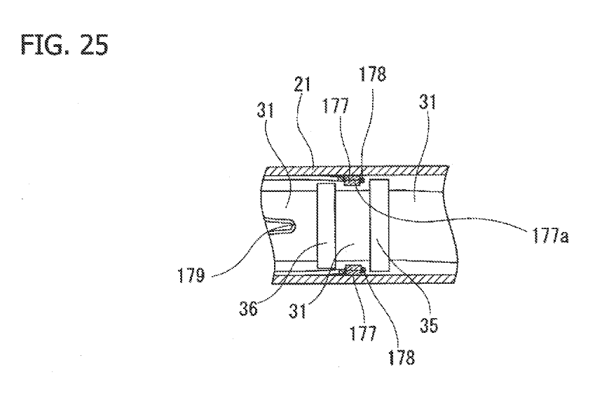

FIG. 25 is an illustration explaining the stent delivery system in which the in-vivo indwelling stent of FIG. 24 is used. As shown in FIG. 25, the stent 170 has the proximal-side inwardly projecting section 177a composed of the radiopaque marker 177. In addition, the stent 170 is so disposed that only proximal-side inwardly projecting sections 177a, namely, only the bent sections 178, are located between the distal-side contact section 36 and the proximal-side contact section 35 of the inner tube body 3. The bent sections 179 each disposed between adjacent bent sections 178 are located on the distal side relative to the distal-side contact section 36. Such a configuration helps ensure that, when the stent 170 is compressed inwardly toward its center axis, the adjacent radiopaque markers 177 are prevented from making contact each other or overlapping with each other, so that good compression is achieved. In addition, it is also relatively easy to dispose the proximal-side inwardly projecting sections 177a of the stent between the distal-side contact section 36 and the proximal-side contact section 35.

The stent delivery system disclosed here by way of several examples of disclosed embodiments comprises a stent delivery system main body including a stent having a multiplicity of side-wall openings, formed in a substantially cylindrical shape, compressed toward its center axis at the time of insertion into a living body, and restorable to its pre-compression shape by expanding outward at the time of indwelling in the living body, an inner tube body having a guide wire lumen, and a stent-accommodating tube body accommodating the stent in a distal end portion thereof, the stent being so disposed as to cover a distal end portion of the inner tube body, and the stent being releasable by moving the stent-accommodating tube body in a proximal direction relative to the inner tube body; and an operation unit disposed at a proximal end portion of the stent delivery system main body and having a moving mechanism for moving the stent-accommodating tube body. The inner tube body includes a distal-side tube having the guide wire lumen, and a proximal-side tube connected to a proximal end side of the distal-side tube and penetrating the stent-accommodating tube body. The operation unit includes a housing, a shaft-like rack member accommodated in the housing and fixed to a proximal end of the stent-accommodating tube body, an operation rotary roller having a working gear wheel which engages with teeth of the rack member so as to move the rack member within the housing, and a connector which is fixed to a proximal end portion of the proximal-side tube protruding beyond the proximal end of the stent-accommodating tube body fixed to the rack member and which is held by the housing. The stent delivery system exhibits stent-holding for releasably holding the stent and for enabling re-accommodation of the stent into the stent-accommodating tube body by forward movement of the stent-accommodating tube body after partial exposure of the stent from the stent-accommodating tube body. The stent can be released from the stent-accommodating tube body by movement of the rack member toward the connector by rotation of the operation rotary roller in a predetermined direction, and, after partial exposure of the stent from the stent-accommodating tube body, the stent can be re-accommodated into the stent-accommodating tube body by moving the rack member within the housing in a direction opposite to a direction toward the connector through rotating the operation rotary roller in a direction reverse to the predetermined direction.

Other aspects of the stent delivery system include the proximal-side tube having a lumen whose distal end portion opens in the stent-accommodating tube body and which provides communication to a proximal end of the proximal-side tube. Liquid can be injected into the stent delivery system from the connector by using the lumen inside the proximal-side tube. The stent includes a distal end portion oriented toward a distal end of the stent-accommodating tube body and a proximal end portion oriented toward the proximal end of the stent-accommodating tube body, and the stent does not have any proximally oriented bent free end other than its proximal end portion, and, after exposure of a distal-side portion from the stent-accommodating tube body, the exposed portion can be re-accommodated into the stent-accommodating tube body by moving the stent-accommodating tube body.

The operation unit has a lock mechanism by which the rack member is releasably locked. The operation unit also has a movement-restraining section which, at the time of rotation of the operation rotary roller in the predetermined direction, makes contact with an end portion of the rack member to thereby restrain the rack member from being moved in excess of a predetermined extent. The operation unit has the movement-restraining section which, at the time of rotation of the operation rotary roller in the direction reverse to the predetermined direction, makes contact with an end portion of the rack member to thereby restrain the rack from being moved in excess of a predetermined extent. The operation unit has a roller intermittent rotation mechanism which imparts a rotation resistance to the operation rotary roller and enables intermittent rotation of the roller.

The stent-holding aspect includes a distal-side contact section as such a portion of the inner tube body as to be located inside the proximal end portion of the stent and as not to enter the side-wall openings of the stent, and a proximal-side contact section as such a portion of the inner tube body as to be located rearward of the proximal end of the stent in proximity to the distal-side contact section, the proximal-side contact section being able to make contact with the proximal end of the stent. The stent-holding aspect can also be a proximal-side inwardly projecting section contactable with the distal-side contact section, the proximal-side inwardly projecting section being located between the distal-side contact section and the proximal-side contact section of the inner tube body. The stent-holding aspect can also include an elastic member which is disposed on the inner tube body at such a position as to be at least in the proximal end portion of the stent and which presses the stent toward the stent-accommodating tube body, and the stent is gripped between the elastic member and the stent-accommodating tube body and is slidable relative to the stent-accommodating tube body.

The detailed description above describes features, aspects and operational characteristics of embodiments of a stent delivery system disclosed here as examples. The invention is not limited, however, to the precise embodiments and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A stent delivery system comprising:
a distal-side tube possessing a guide wire lumen which opens at opposite ends to permit passage of a guide wire to guide the stent delivery system to a target site in a living body, the distal-side tube possessing a proximal end portion;
a proximal-side tube connected to the proximal end portion of the distal-side tube, the proximal-side tube possessing a distal end portion;
a stent-accommodating tube body surrounding at least a portion of the distal-side tube and the distal end portion of the proximal-side tube, the stent-accommodating tube body possessing a distal end portion having an inner surface spaced outwardly from an outer surface of the portion of the distal-side tube so that a space exists between the outer surface of the portion of the distal-side tube and the inner surface of the distal end portion of the stent-accommodating tube body;
a stent accommodated in the space between the outer surface of the portion of the distal-side tube and the inner surface of the distal end portion of the stent-accommodating tube body so that the stent surrounds the portion of the distal-side tube and is covered by the distal end portion of the stent-accommodating tube body;
the stent including a side-wall provided with a plurality of through openings, the stent being compressed inwardly while accommodated in the space and covered by the distal end portion of the stent-accommodating tube body and being restorable to a pre-compression shape by expanding outwardly when the stent-accommodating tube is moved proximally relative to the distal-side tube to release the stent;
an elongated rack member positioned in a housing and fixed to a proximal end of the stent-accommodating tube body;
a rotatably mounted operation roller operatively engaging the rack so that operative rotation of the operation roller moves the rack member relative to the housing to thus move the stent-accommodating tube body; and
a stent holder positioned in the space between the outer surface of the portion of the distal-side tube and the inner surface of the distal end portion of the stent-accommodating tube body, the stent holder holding the proximal end portion of the stent so that when a distal end portion of the stent is exposed outside the stent-accommodating tube body and is no longer covered by the stent-accommodating tube body by virtue of the stent-accommodating tube body being moved in a proximal direction relative to the distal-side tube through rotation of the operation roller in one rotational direction, the exposed distal end portion of the stent is re-accommodated inside and covered by the distal end portion of the stent-accommodating tube body through rotation of the roller in a rotational direction opposite the one rotational direction.

2. The stent delivery system according to claim 1, wherein at least a portion of the stent holder axially overlaps the proximal end portion of the stent.

3. The stent delivery system according to claim 1, wherein the stent holder comprises two spaced apart contact sections which project away from the outer surface of the portion of the distal-side tube.

4. The stent delivery system according to claim 3, wherein the proximal end portion of the stent is positioned between the two spaced apart contact sections.

5. The stent delivery system according to claim 1, wherein the stent holder comprises a contact section on the outer surface of the portion of the distal-side tube and projecting away from the outer surface of the portion of the distal-side tube, the contact section being positioned distally of a proximal-most end of the stent and proximally of a distal-most end of the stent.

6. The stent delivery system according to claim 1, wherein the stent holder encircles a portion of an axial extent of the distal-side tube.

7. The stent delivery system according to claim 1, wherein the stent holder is annular-shaped.

8. The stent delivery system according to claim 1, wherein the stent holder includes an elastic member surrounding the distal-side tube and contacting an inner surface of the proximal end portion of the stent.

9. The stent delivery system according to claim 1, wherein the proximal end portion of the stent includes an inwardly projecting section that projects inwardly toward the outer surface of the portion of the distal-side tube, and the stent holder comprises two spaced apart contact sections which project away from the outer surface of the portion of the distal-side tube, the inwardly projecting section of the proximal end portion of the stent being positioned between the two spaced apart contact sections.

10. A stent delivery system comprising:
a stent delivery system main body and an operation unit, the operation unit being disposed at a proximal end portion of the stent delivery system main body, the stent delivery system main body including:
a substantially cylindrically-shaped stent possessing a center axis and having a multiplicity of side-wall openings, the stent being compressed toward its center axis upon insertion into a living body and being restorable to its pre-compression shape by expanding outward during indwelling in the living body, the stent possessing a proximal end portion;
an inner tube body possessing a distal end portion, the inner tube body including a distal-side tube having a guide wire lumen, and a proximal-side tube connected to a proximal end portion of the distal-side tube;
a stent-accommodating tube body possessing a distal end portion, the proximal-side tube penetrating the stent-accommodating tube body, the stent being accommodated in the distal end portion of the stent-accommodating tube body;
the stent covering the distal end portion of the inner tube body;
the stent being releasable by moving the stent-accommodating tube body in a proximal direction relative to the inner tube body; and
the operation unit including:
a housing;
a shaft-shaped rack member accommodated in the housing and fixed to a proximal end of the stent-accommodating tube body, the rack member possessing teeth;
a rotatably mounted operation rotary roller having a working gear wheel which engages the teeth of the rack member to move the rack member within the housing; and
a connector fixed to a proximal end portion of the proximal-side tube and protruding proximally beyond the proximal end of the stent-accommodating tube body, the connector being held by the housing, and
means for releasably holding the proximal end portion of the stent to permit re-accommodation of the stent into the stent-accommodating tube body by forward movement of the stent-accommodating tube body after partial exposure of the stent from the stent-accommodating tube body, the stent being releasable from the stent-accommodating tube body by moving the rack member toward the connector through rotation of the operation rotary roller in one rotational direction and, after partial exposure of the stent from the stent-accommodating tube body, the stent is re-accommodated into the stent-accommodating tube body by moving the rack member within the housing away from the connector through rotation of the operation rotary roller in a direction reverse to the one rotational direction.

11. The stent delivery system according to claim 10, wherein the proximal-side tube possesses a lumen having a distal end which opens into the stent-accommodating tube body, the lumen in the proximal-side tube communicating with a proximal end of the proximal-side tube.

12. The stent delivery system according to claim 11, wherein liquid is injectable into the stent delivery system from the connector by way of the lumen in the proximal-side tube.

13. The stent delivery system according to claim 10, wherein the stent includes a distal end portion oriented toward a distal end of the stent-accommodating tube body and a proximal end portion oriented toward the proximal end of the stent-accommodating tube body, the stent being devoid of any proximally oriented bent free end other than the proximal end portion of the stent, and after exposure of a distal end portion of the stent from the stent-accommodating tube body, the exposed distal end portion is re-accommodated into the stent-accommodating tube body by moving the stent-accommodating tube body distally.

14. The stent delivery system according to claim 10, wherein the operation unit includes a lock mechanism which releasably locks the rack member.

15. The stent delivery system according to claim 10, wherein the operation unit includes a movement-restraining section which, during rotation of the operation rotary roller in the one rotational direction, contacts an end portion of the rack member to restrain the rack member from being moved in excess of a first predetermined extent.

16. The stent delivery system according to claim 15, wherein the operation unit includes the movement-restraining section which, during rotation of the operation rotary roller in the direction reverse to the one rotational direction, contacts an end portion of the rack member to thereby restrain the rack member from being moved in excess of a second predetermined extent.

17. The stent delivery system according to claim 10, wherein the operation unit includes a roller intermittent rotation mechanism which imparts a rotation resistance to the operation rotary roller and enables intermittent rotation of the roller.

18. The stent delivery system according to claim 10, wherein the means for releasably holding the proximal end portion of the stent includes a distal-side contact section and a proximal-side contact section which are both fixed to the inner tube body, the distal-side contact section being positioned distally of the proximal end portion of the stent and configured so that the distal-side contact section does not enter the side-wall openings of the stent, the proximal-side contact section being proximally spaced from the distal-side contact section, the stent possessing a proximal-most end positioned between the distal-side contact section and the proximal-side contact section, and the proximal-side contact section being contactable with the proximal end of the stent.

19. The stent delivery system according to claim 18, wherein as a part of the means for releasably holding the proximal end portion of the stent, the stent includes a proximal-end inwardly projecting section projecting inwardly toward the distal-side tube and being contactable with the distal-side contact section, the proximal-end inwardly projecting section being located between the distal-side contact section and the proximal-side contact section.

20. The stent delivery system according to claim 10, wherein the means for releasably holding the proximal end portion of the stent includes an elastic member disposed on the inner tube body at a position axially overlapping with at least a proximal end portion of the stent and which presses the stent outwardly toward the stent-accommodating tube body so that the stent is gripped between the elastic member and the stent-accommodating tube body and is slidable relative to the stent-accommodating tube body.
